(19) United States
(12) Patent Application Publication
(10) Pub. No.: US 2005/0222665 A1
(43) Pub. Date: Oct. 6, 2005

(54) Endovascular fastener applicator

(76) Inventor: Ernest Aranyi, Easton, CT (US)

Correspondence Address: UNITED STATES SURGICAL,;A DIVISION OF TYCO HEALTHCARE GROUP LP, 150 GLOVER AVENUE, NORWALK, CT, 06856, US

(21) Appl. No.: 11/100,900

(22) Filed: Apr. 7, 2005

Related U.S. Application Data
(63) Continuation-in-part of application No. 10/053,889, filed on Oct. 23, 2001.
Application No. 10/053,889 is a continuation of application No. PCT/US00/10921, filed on Apr. 21, 2000.
(60) Provisional application No. 60/130,922, filed on Apr. 23, 1999.

(51) Int. Cl. A61F 2/06; A61B 17/10; A61B 17/08

(52) U.S. Cl. 623/1.11; 606/142

(57) ABSTRACT

Endovascular fastener applicator for endoluminally fastening prosthetic grafts to vessels, are provided. The endovascular fastener applicator includes a delivery assembly configured for positioning within a vessel, and a control assembly mounted to a proximal end of the outer sheath for extracorporeal control of the delivery assembly. The delivery assembly includes an expandable portion disposed adjacent a distal end of an outer sheath and being expandable to support a prosthetic in contact with an inner surface of a vessel; a yoke assembly disposed within the expandable portion; an applicator head assembly pivotably mounted to the yoke assembly and movable between a loading position longitudinally aligned with the yoke assembly, and a firing position oriented substantially perpendicular to the yoke assembly; and a fastener assembly connectable to a distal end of the expandable portion, the fastener assembly retaining at least one fastener therein.

### CROSS-REFERENCE TO RELATED APPLICATIONS

The present application is a continuation-in-part application which claims benefit of and priority to U.S. patent application Ser. No. 10/053,889, filed on Oct. 23, 2001 which claims the benefit of and priority to International Application No. PCT/US00/10921 filed on Apr. 21, 2000 which in turn claims the benefit of and priority to U.S. Provisional Application Ser. No. 60/130,922 filed Apr. 23, 1999, the entire contents of each of which are hereby incorporated in their entirety by reference.

### BACKGROUND

1. Technical Field

This disclosure relates generally to vascular grafts for intraluminal delivery, and in particular, to apparatus and methods for repairing diseased or damaged sections of a vessel by fastening a prosthesis within the vessel.

2. Description of Related Art

Diseased or damaged blood vessels often cause weakening of the vessel wall resulting in an aneurysm whereby a blood vessel and especially an artery have a section of abnormal blood-filled dilation. For example, an abdominal aortic aneurysm is a sac caused by an abnormal dilation of the wall of the aorta, a major artery of the body, as it passes through the abdomen.

The abdominal aortic aneurysm usually arises in the infrarenal portion of the arteriosclerotically diseased aorta, for example, below the kidneys. Left untreated, the aneurysm will eventually cause nipture of the sac with ensuing fatal hemorrhaging in a very short time. High mortality associated with rupturing led the state of the art into trans-abdominal surgical repair of abdominal aortic aneurysms.

Surgery involving the abdominal wall, however, is a major undertaking with associated high risks. This type of surgery, in essence, involves replacing the diseased and aneurismal segment of blood vessel with a prosthetic device which typically is a synthetic tube, or graft, usually fabricated of either DACRON® polyester, TEFLON® fluoropolymer resin available from E.I. du Pont de Nemours and Company, or other suitable material.

The present state of the art for intraluminal repair of a vessel does not fasten a prosthesis to the remaining aortic wall. For example, U.S. Pat. Nos. 5,571,171 and 5,571,173 disclose a method and apparatus for treating an abdominal aortic aneurysm by supplying a prosthesis or an aortic graft for intraluminal delivery that does not fasten the graft to the remaining aortic wall.

Presenting an aortic graft through the aorta by intraluminal delivery avoids major invasive surgery. The '171 and '173 patents disclose an aortic graft that is delivered intraluminally to the aneurysm site. The aortic graft is secured to the remaining aortic wall by a balloon that is inflated thereby causing the graft to contact and adhere to the remaining aortic wall.

The major disadvantages related to the combination of endovascular expanders, such as a balloon or stent, and prosthesis is the dilation of the natural artery with consequent migrations and periprosthetic losses. Upon withdrawal of the expander, the tissue is caused to collapse and the prosthesis disengages from the remaining aortic wall and tends to migrate to a location away from the aneurysm site to be repaired. The migration and movement of the disengaged aortic graft would then obstruct the affected vessel. The migration and movement of the aortic graft requires further treatment on the patient to remove the failed attempt to attach the aortic graft to the remaining aortic wall.

Further treatment may include major surgery that is hazardous and traumatic to the patient. Major surgery to remove the aortic graft defeats the benefits of intraluminal delivery of the aortic graft. The current state of the art does not disclose a fastener applicator that intraluminally delivers a vascular graft and endoluminally applies internal fasteners to fasten a prosthesis in place.

Accordingly, there is a present need for a fastener applicator that intraluminally delivers a vascular graft to a site within a vessel and applies fasteners to pass through both a prosthesis and the thickness of a vessel wall. The fastened prosthesis should also have the capability of following dilation of a vessel.

### SUMMARY

According to an aspect of the present disclosure, an endovascular fastener applicator for endoluminally fastening a prosthetic graft to a vessel with at least one fastener, is provided. The endovascular fastener applicator includes a delivery assembly configured for positioning within a vessel, and a control assembly mounted to a proximal end of the outer sheath for extracorporeal control of the delivery assembly. The delivery assembly includes an expandable portion disposed adjacent a distal end of an outer sheath and being expandable to support a prosthetic in contact with an inner surface of a vessel; a yoke assembly disposed within the expandable portion; an applicator head assembly pivotably mounted to the yoke assembly and movable between a loading position longitudinally aligned with the yoke assembly, and a firing position oriented off axis to the yoke assembly; and a fastener assembly positioned at a distal end of the expandable portion, wherein the fastener assembly retaining at least one fastener therein.

The control assembly has a first knob to expand the expandable portion. The control assembly also includes a second knob to pivot the applicator head assembly from the loading position to the firing position. The control assembly further includes a third knob to rotate the applicator head assembly about a longitudinal axis of the outer sheath. Desirably, the second knob of the control assembly withdraws a fastener out of the fastener assembly and drives the fastener into tissue. It is envisioned that the fastener is a helical coil fastener.

According to another aspect of the present disclosure, an endovascular fastener applicator for endoluminally fastening a prosthetic graft to a vessel with at least one fastener is provided. The endovascular fastener applicator includes an outer sheath; a delivery assembly for delivering a graft to a site within a vessel and for fastening a prosthetic graft to a vessel by passing a fastener therethrough; and a control assembly mounted to a proximal end of the outer sheath for extracorporeal control of the delivery assembly.

The delivery assembly includes an expandable portion disposed adjacent a distal end of the outer sheath and deployable to support a prosthetic in contact with an inner surface of a vessel; a drive assembly operatively disposed within the expandable portion; and a fastener assembly positioned at a distal end of the expandable portion, wherein the fastener assembly retaining at least one fastener therein.

The drive assembly includes a yoke assembly for guiding and supporting an applicator head assembly; and an applicator head assembly pivotably mounted to the yoke assembly and movable between a loading position longitudinally aligned with the yoke assembly, and a firing position oriented substantially perpendicular to the yoke assembly.

The fastener assembly includes a body portion; a central shaft rotatably disposed within the body portion; and a coil disposed between the body portion and the central shaft configured to operatively engage a coil of a helical fastener. The central shaft defines an elongate slot extending along the length thereof for receiving a tang of a helical fastener therein, the central shaft defining a recess formed in a proximal end thereof for selectively receiving a distal end of a drive shaft therein.

The expandable portion includes a proximal hub and a distal hub; and a plurality of support members extending between and inter-connecting the proximal and distal hubs. The expandable portion having a un-expanded condition wherein the proximal and distal hubs are in relative spaced relation to one another and the support members in relative close proximity to one another, and an expanded condition wherein the proximal and distal hubs are in relative close proximity to one another and the support members radially expand to define interstitial regions therebetween.

The drive assembly is rotatable relative to the expandable portion. The applicator head assembly is pivotable between an orientation axially aligned with a longitudinal axis of the yoke assembly and an orientation perpendicular to the longitudinal axis of the yoke assembly.

In one embodiment, the yoke assembly includes a body portion having a pair of elongate slots formed on either side thereof, the applicator head assembly being operatively disposed within the body portion of the yoke assembly; a pivot pin extending through the body portion and the applicator head assembly, at a location adjacent to the elongate slots; and a clevis operatively disposed within the body portion of the yoke assembly.

The clevis includes a pair of spaced apart arms; and a cam pin extending from each arm of the clevis and slidably engaging a respective elongate slot of the body portion. Each cam pin slidably engaging a cam slot formed in the applicator head assembly.

The yoke assembly further includes a driver operatively connected to the clevis; and a pusher operatively engaged with the base portion of the clevis and including a body portion defining a lumen configured to receive the elongate body portion of the driver. The drive may include a coupling member configured to selectively engage a shaped end of the drive screw and a shaped end of a second bevel gear; and an elongate body portion extending from the coupling member and extending through an aperture formed in a base portion of the clevis.

The applicator head assembly includes a housing defining a cam slot formed on either side thereof, each cam slot being configured to receive a respective can pin of the yoke assembly; and a drive train operatively disposed in the housing. Each cam slot includes a first portion substantially aligned with a longitudinal axis of the housing; and a second portion substantially perpendicular to the longitudinal axis of the housing.

The drive train of the applicator head assembly includes a drive shaft having a distal end configured to selectively receive a tang of a helical fastener, and a non-circular transverse cross-sectional profile; and a drive screw slidably disposed on a proximal end portion of the drive shaft. The drive screw includes a helical thread configured to engage threads formed in the housing, and a shaped proximal end configured to selectively engage the coupling member of the driver of the yoke assembly when the applicator head assembly is in the loading position.

The drive train further includes a beveled gear system. The beveled gear system includes a first beveled gear co-axially aligned with and slidably supported on the drive shaft of the drive train; a gear support box slidably and rotatably supported on the drive shaft and including a stub extending therefrom; and a second beveled gear rotatably supported on the stub of the gear support box and operatively engaged with the first beveled gear. The first beveled gear includes a shaped central lumen configured to complement the cross-sectional profile of the drive shaft of the drive train. The second beveled gear includes a shaped end configured to selectively engage the coupling member of the driver of the yoke assembly when the applicator head assembly in is the firing position.

The applicator head assembly includes an ejection head operatively positioned on a distal end portion of the drive shaft, the ejection head includes a central lumen defining a helical thread formed at a distal end thereof and a recess formed in a proximal edge thereof; and an ejection head stop operatively positioned within the ejection head and supported on the distal end portion of the drive shaft. The ejection head stop includes a key extending therefrom and configured for engagement in the recess formed in the proximal edge of the ejection head and a slot formed in the housing of the applicator assembly.

The ejection head includes a saw-toothed distal end face for gripping and engaging a prosthetic graft. The applicator head assembly includes a coil spring supported on the drive shaft and positioned between the drive screw and the gear support box, wherein the coil spring biases the drive shaft to a proximal-most position. The pivot pin fixes the axial location of the gear support box relative to the housing.

The control assembly includes a housing for supporting an expandable portion controller and a drive assembly controller; an expandable portion controller configured to manipulate the expandable portion between the un-expanded condition and the expanded condition; and a drive assembly controller configured to manipulate the applicator head assembly between loading position and the firing position, and to rotate the applicator head assembly about a longitudinal axis of the yoke assembly.

The expandable portion controller includes a knob rotatably supported in the housing thereof; a spool in threaded engagement with the knob; an outer sheath having a distal end fixedly secured to the proximal hub of the expandable portion, and a proximal end fixedly secured to the spool; a flange fixedly supported in the housing of the control assembly; and a first inner tube extending through the spool and through the outer sheath. The first inner tube has a proximal end fixedly secured to the flange, and a distal end secured to the body portion of the yoke assembly. Accordingly, as the knob is rotated relative to spool, the spool axially translates through the knob causing outer sheath to displace relative to the first inner tube thereby manipulating the expandable portion between the un-expanded condition and the expanded condition.

The drive assembly controller includes a knob rotatably supported in the housing of the control assembly; and a second inner tube extending through the first inner tube. The second inner tube includes a proximal end fixedly secured to the knob of the drive assembly controller, and a distal end operatively connected to the body portion of the pusher of the yoke assembly. Accordingly, rotation of the knob of the drive assembly controller manipulates the applicator head assembly between the loading position and the firing position.

The drive assembly controller further includes a drive handle rotatably and translatably supported on the hosing of the control assembly; and a drive shaft extending through the second inner tube and the body portion of the pusher. The drive shaft includes a proximal end fixedly connected to the drive handle and a distal end fixedly connected to the body portion of the driver of the yoke assembly. Accordingly, as drive handle is translated relative to the housing of the control assembly the driver of concomitantly translated to selectively engage and disengage the coupling member of the driver with the shaped proximal end of the drive screw and the shaped end of the second bevel gear. Additionally, when the coupling member is engaged with the shaped proximal end of the drive screw or the shaped end of the second bevel gear, rotation of the drive handle results in rotation of the drive screw or the second bevel gear, respectively. It is envisioned that rotation of the drive screw or the second bevel gear results in rotation of the drive shaft.

The fastener applicator may further include a cap operatively connectable to a distal end of the fastener cartridge assembly. The cap includes a lumen having a distal end extending through a distal end of the cap and a proximal end extending through a side of the cap. It is envisioned that the lumen is configured and dimensioned to slidably receive a guide wire.

According to another aspect of the present disclosure, a method of endoluminally fastening a prosthetic graft to a vessel, is provided. The method includes the steps of providing an endovascular fastener applicator for endoluminally fastening the prosthetic graft to a vessel at the aneurysm site. The endovascular fastener applicator includes a delivery assembly configured for positioning within a vessel. The delivery assembly includes an expandable portion disposed adjacent a distal end of an outer sheath and being expandable to support the prosthetic graft in contact with an inner surface of the vessel; a yoke assembly disposed within the expandable portion; an applicator head assembly pivotably mounted to the yoke assembly and movable between a loading position longitudinally aligned with the yoke assembly, and a firing position oriented substantially perpendicular to the yoke assembly; and a fastener assembly positioned at a distal end of the expandable portion. The fastener assembly retains at least one fastener therein for loading onto the applicator head assembly when the applicator head assembly is in the longitudinally aligned position. The fastener applicator further includes a control assembly mounted to a proximal end of the outer sheath for extracorporeal control of the delivery assembly.

The method further includes the steps of positioning a prosthetic graft at an aneurysm site; positioning the delivery assembly at the aneurysm site; radially expanding the expandable portion against an inner surface of the prosthetic graft to thereby maintain the prosthetic graft in position at the aneurysm site; connecting the applicator head assembly to the fastener assembly; transferring a helical fastener from the fastener assembly to the applicator head assembly; disconnecting the applicator head assembly from the fastener cartridge assembly; pivoting the applicator head assembly from the loading position to the firing position; and firing the applicator head assembly and driving the helical fastener loaded thereon into the prosthetic graft.

### BRIEF DESCRIPTION OF THE-DRAWINGS

Various embodiments are described herein with reference to the drawings, wherein.

FIG. 1 is a perspective view of an endovascular fastener applicator in accordance with an embodiment of the present disclosure with a delivery assembly in an extended condition;

FIG. 2 is a perspective view of the endovascular fastener applicator of FIG. 1 with the delivery assembly in a bent condition;

FIG. 3 is a cross-sectional view, in part elevation, of an aortic graft placed at the site of an abdominal aortic aneurysm within the aorta;

FIG. 4 is an enlarged detail view of a portion of FIG. 3 illustrating the aortic graft in partial cut-away secured to the remaining aortic wall and maintained in position by helical fasteners;

FIG. 5 is a cross-sectional view, in part elevation, of an aortic graft for treating an aortic aneurysm affecting the aorta and both ileac arteries;

FIG. 6 is a perspective view, with parts separated, of a fastener cartridge assembly and drive assembly of the endovascular fastener applicator of FIGS. 1 and 2;

FIG. 7 is a perspective view of a driver of the drive assembly of FIG. 6;

FIG. 8 is an end, perspective view of a pusher of the drive assembly of FIG. 6;

FIG. 9 is a perspective view, with parts separated, of the fastener cartridge assembly of FIG. 6;

FIG. 10 is an enlarged end view of a central shaft of the fastener cartridge assembly of FIG. 9 with a coil fastener shown operatively disposed thereon;

FIG. 11 is a perspective view of the coil fastener shown in FIG. 10;

FIG. 12 is a perspective view of an expandable portion of the drive assembly of FIG. 6;

FIG. 13 is a perspective view, with parts separated, of the expandable portion of FIG. 12;

FIG. 14 is a further perspective view, with parts separated, of the expandable portion of FIGS. 12 and 13;

FIG. 15 is a perspective view of a yoke assembly of the drive assembly of FIG. 6;

FIG. 16 is a perspective view, with parts separated, of an arm of the yoke assembly of FIG. 6;

FIG. 17 is a perspective view of the arm of FIG. 16;

FIG. 18 is a bottom perspective view of the yoke assembly of FIG. 15 with an applicator head assembly, according to the present disclosure, operatively associated therewith;

FIG. 19 is a top, front perspective view of the yoke assembly and applicator head assembly of FIG. 18;

FIG. 20 is a top, rear perspective view of the yoke assembly and applicator head assembly of FIGS. 18 and 19

FIG. 21 is a top, rear perspective view of the applicator head assembly of the present disclosure;

FIG. 22 is a bottom, front perspective view of the applicator head assembly of FIG. 21;

FIG. 23 is a top, rear perspective view of the applicator head assembly of FIGS. 21 and 22, with the housing removed therefrom;

FIG. 24 is a bottom, front perspective view of the applicator head assembly of FIGS. 21 and 22, with the housing removed therefrom;

FIG. 25 is a longitudinal cross-sectional view of the applicator head assembly of FIG. 23 as taken through 25-25;

FIG. 26 is a longitudinal cross-sectional view of the applicator head assembly of FIG. 21 as taken through 26-26;

FIG. 27 is a top, rear perspective view, with parts separated, of the applicator head assembly of FIGS. 21-26;

FIG. 28 is a perspective view of a drive shaft of the applicator head assembly of FIG. 27;

FIG. 29 is a top, rear perspective view, of a control assembly according to the present disclosure, with a half-section removed therefrom;

FIG. 30 is a top, front perspective view, of the control assembly as shown in FIG. 29;

FIG. 31 is a cross-sectional view of the elongated tube of the fastener applicator, as taken through 31-31 of FIG. 2;

FIG. 32 is a perspective view, with parts separated, of the control assembly of FIGS. 29 and 30;

FIG. 33 is a longitudinal cross-sectional view of the fastener applicator of FIG. 1, as taken through 33-33 of FIG. 1;

FIG. 34 is a longitudinal cross-sectional view of the fastener applicator of FIG. 1, as taken through 34-34 of FIG. 1;

FIG. 35 is an enlarged view of the area indicated as 35 of FIG. 33;

FIG. 36 is an enlarged view of the area indicated as 36 of FIG. 34;

FIG. 37 is an enlarged view of the area indicated as 37 of FIG. 33;

FIG. 38 is an enlarged view of the area indicated as 38 of FIG. 34;

FIG. 39 is an enlarged view of the area indicated as 39 of FIG. 37;

FIG. 40 is an enlarged view of the area indicated as 40 of FIG. 38;

FIG. 41 is an enlarged view of the area indicated as 41 of FIG. 33;

FIG. 42 is an enlarged view of the area indicated as 42 of FIG. 34;

FIG. 43 is a cross-sectional view, in part elevation, of the abdominal aortic aneurysm of FIG. 3, with an aortic graft placed at therein and with the distal end of the fastener applicator of FIGS. 1 and 2 operatively disposed therethrough such that the expandable portion is radially aligned with a reinforcing band of the aortic band;

FIG. 44 is a cross-sectional view of the control assembly of FIGS. 29 and 30, illustrating the operation thereof to expand the expandable portion of the fastener applicator;

FIG. 45 is an enlarged view of the expandable portion of the fastener applicator, in an expanded position, against the inner wall of the aortic graft;

FIG. 46 is a cross-sectional view of the control assembly of FIGS. 29 and 30, illustrating the operation thereof to engage the applicator head assembly with the fastener cartridge assembly;

FIG. 47 is a cross-sectional view of the applicator head assembly and the fastener cartridge assembly, illustrating operative engagement thereof;

FIG. 48 is an enlarged view of the area indicated as 48 of FIG. 47, rotated 90° about the longitudinal axis;

FIG. 49 is a perspective view illustrating the coupling of the drive shaft of the applicator head assembly to the central shaft of the fastener cartridge assembly;

FIG. 50 is a perspective view illustrating the drive shaft of the applicator head assembly coupled to the central shaft of the fastener cartridge assembly;

FIG. 51 is a cross-sectional view of the control assembly of FIGS. 29 and 30, illustrating the operation thereof to load the applicator head assembly with a fastener from the fastener cartridge assembly;

FIG. 52 is a cross-sectional view of the applicator head assembly and the fastener cartridge assembly, illustrating the loading of the fastener on to the applicator head assembly from the fastener cartridge assembly;

FIG. 53 is a cross-sectional view of the control assembly of FIGS. 29 and 30, illustrating the operation thereof to disengage the applicator head assembly from the fastener cartridge assembly;

FIG. 54 is a cross-sectional view of the applicator head assembly and the fastener cartridge assembly, illustrating the disengagement of the applicator head assembly from the fastener cartridge assembly;

FIG. 55 is a cross-sectional view of the control assembly, as taken through 55-55 of FIG. 2, illustrating a safety lock-out in a first position;

FIG. 56 is a cross-sectional view of the control assembly, as taken through 55-55 of FIG. 2, illustrating the safety lock-out in a second position;

FIG. 57 is a cross-sectional view of the control assembly of FIGS. 29 and 30, illustrating the movement of the safety lock-out from the first position to the second position and the operation of the control assembly to tilt the applicator head assembly from a first, substantially axially aligned position, to a second, rotated position;

FIGS. 58-60 are schematic side elevational views of applicator head assembly illustrating the pivoting of the applicator head assembly from the first position to the second position;

FIG. 61 is a cross-sectional view of the control assembly of FIGS. 29 and 30, illustrating the operation thereof to engage the driver with the applicator head assembly in the second position and to the expel the fastener therefrom;

FIG. 62 is a cross-sectional view of the applicator head assembly, illustrating the expelling of the fastener from the applicator head assembly and into the aortic graft and aortic wall;

FIG. 63 is a cross-sectional view of the distal end of the applicator head assembly, illustrating the driving of the fastener loaded thereon into the aortic graft and the aortic wall;

FIG. 64 is a cross-sectional view of the applicator head assembly illustrating disengagement of the applicator head assembly from the surface of the aortic graft;

FIGS. 65-67 are side elevational views of a rotation knob of control assembly, illustrating the operation thereof to rotating the applicator head assembly about the longitudinal axis;

FIG. 68 is a top view of the expandable portion in the expanded position and the applicator head assembly in the second position, illustrating the rotation of the applicator head assembly, about the longitudinal axis relative to the expandable portion;

FIG. 69 is a top, rear perspective view of an applicator head assembly according to an alternate embodiment of the present disclosure;

FIG. 70 is a bottom, front perspective view of the applicator head assembly of FIG. 69; and

FIGS. 71-76 are schematic side elevational views illustrating the pivoting of the applicator head assembly of FIGS. 69 and 70 from the first position to the second position.

### DETAILED DESCRIPTION OF EMBODIMENTS

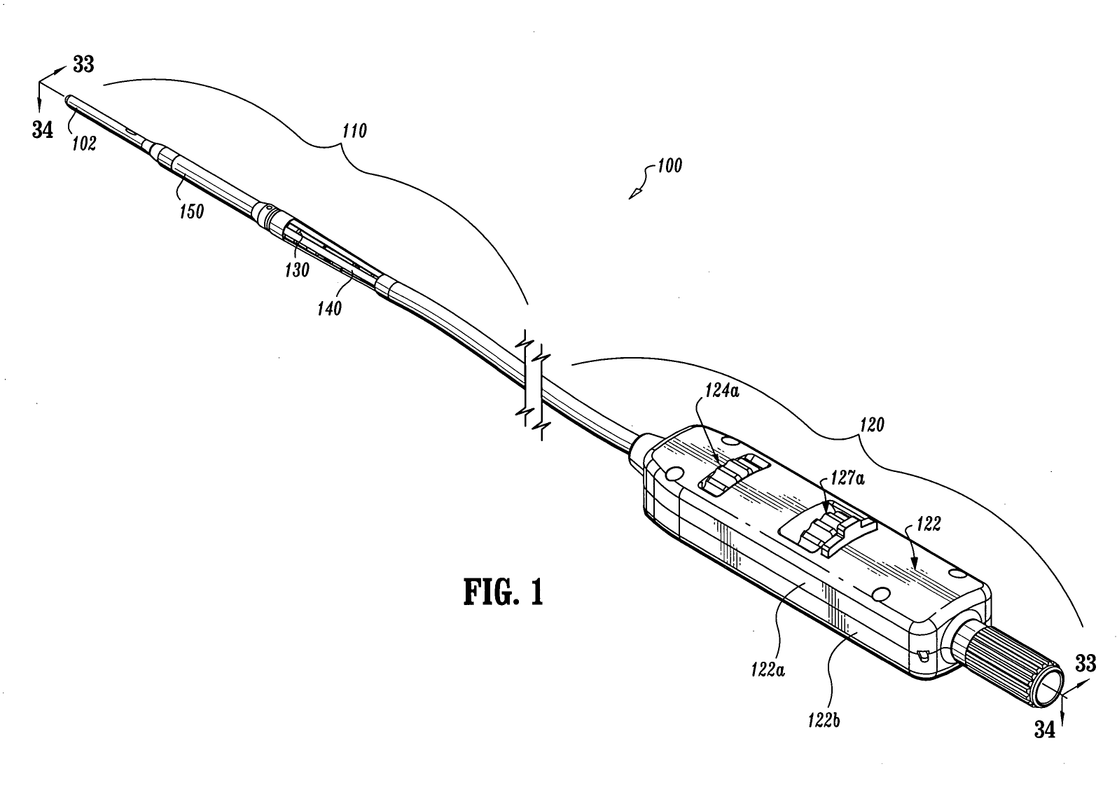

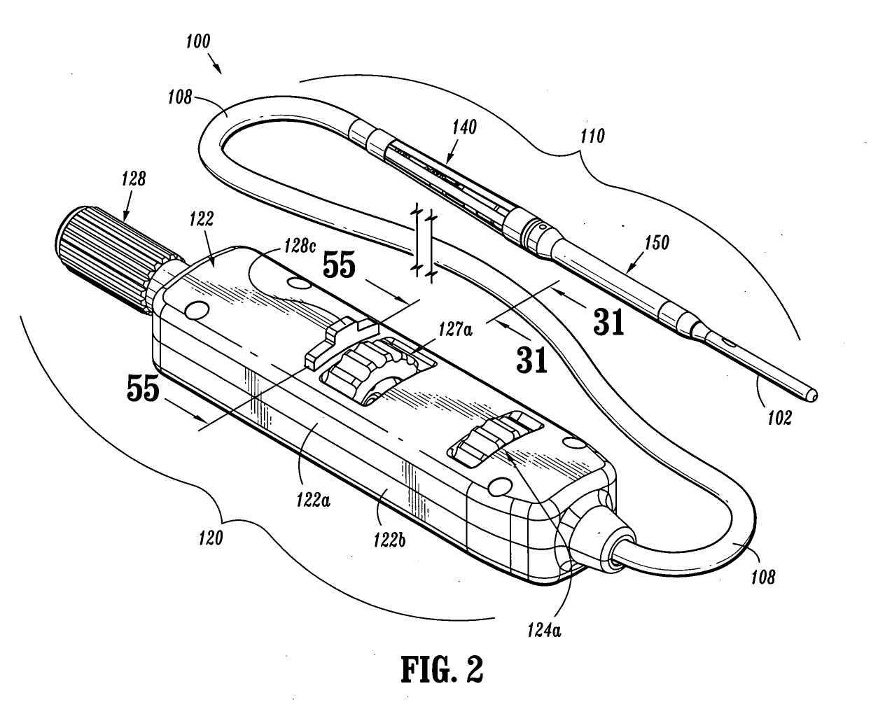

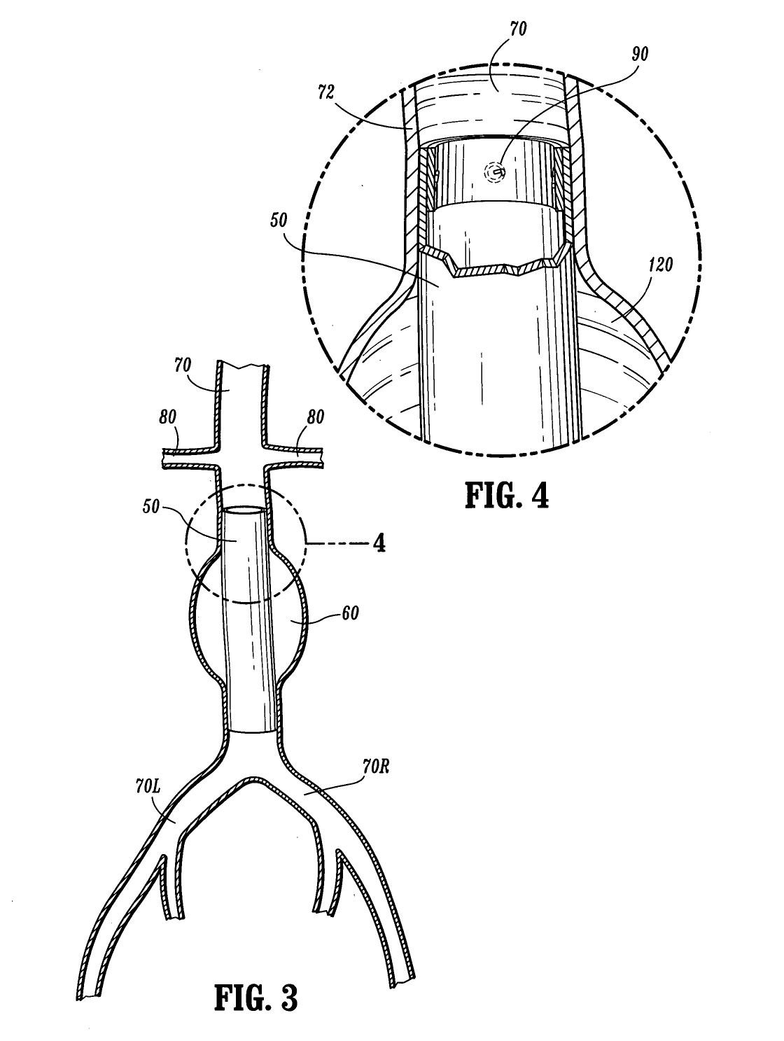

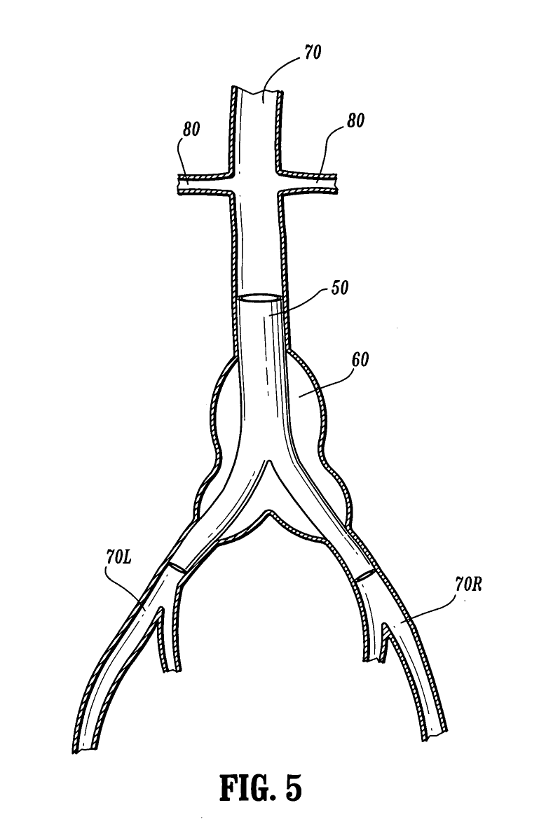

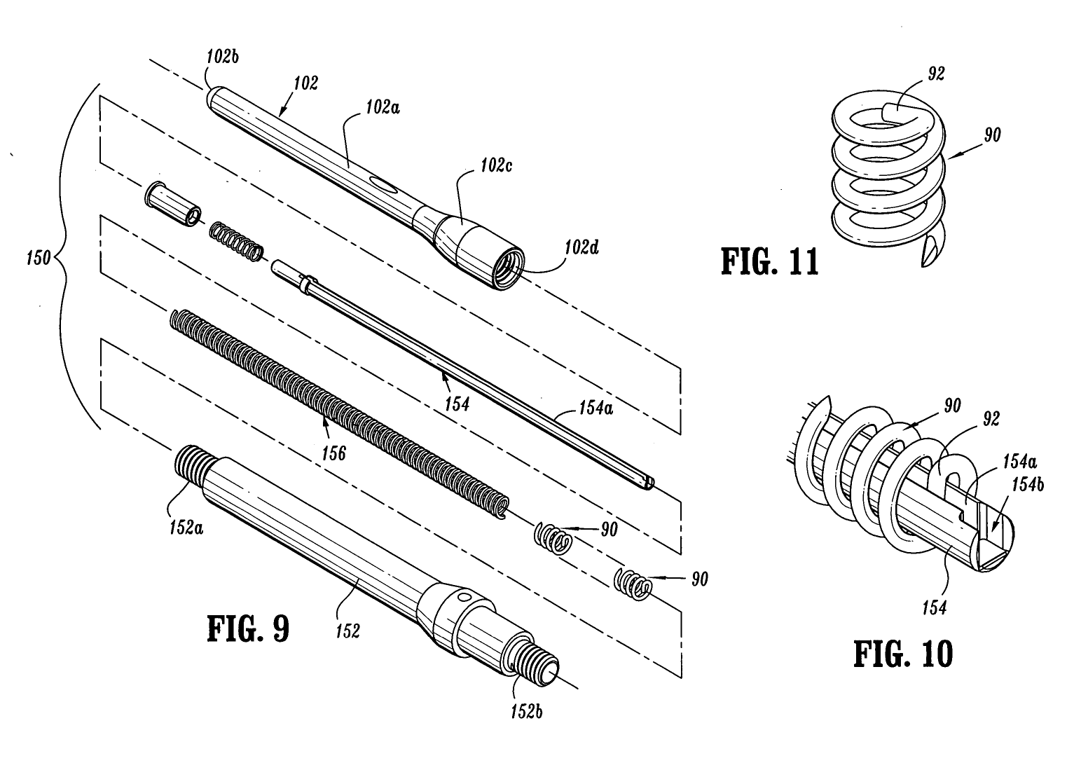

As illustrated in FIGS. 1 and 2, the present disclosure relates to an endovascular fastener applicator, generally referred to as 100. Endovascular fastener applicator 100 includes a delivery assembly 110 and a control assembly 120 operatively connected to delivery assembly 110 by an elongate body portion 108. Endovascular fastener applicator 100 delivers an aortic graft 50, as shown in FIGS. 3-5, for repairing an abdominal aortic aneurysm 60 in aorta 70 having two iliac arteries 70L and 70R associated therewith, as well as a plurality of renal arteries 80 located above aneurysm 60 in fluid communication with aorta 70. Repairing aneurysm 60 includes fastening aortic graft 50 to aortic wall 72 using fasteners 90 (see FIGS. 9-11). Aortic graft 50, as well as other prosthesis, may be utilized in the thoracic aorta, and can be used to repair thoracic aneurysms or thoracic dissecting aneurysms. Further, fastener applicator 100 may also treat vascular trauma and other obstructive diseases with various prosthesis. Accordingly, use of the term aortic aneurysm in the specification and claims is intended to relate to and mean both abdominal aortic aneurysms, thoracic aneurysms and related vessel diseases. Delivery assembly 110 includes a drive assembly 130, an expandable portion 140 operatively connected to drive assembly 130, a fastener cartridge assembly 150 operatively connected to a distal end of drive assembly 130 and expandable portion 140, and a cap 102 operatively connectable to a distal end of fastener cartridge assembly 150.

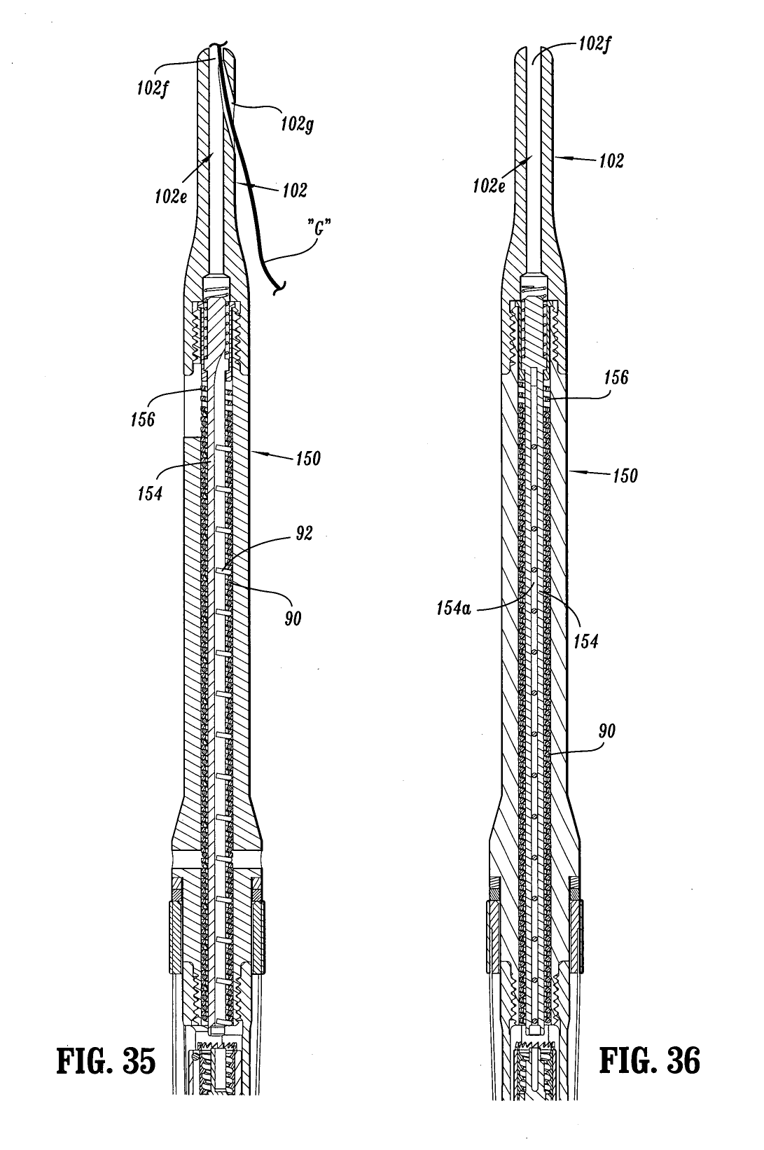

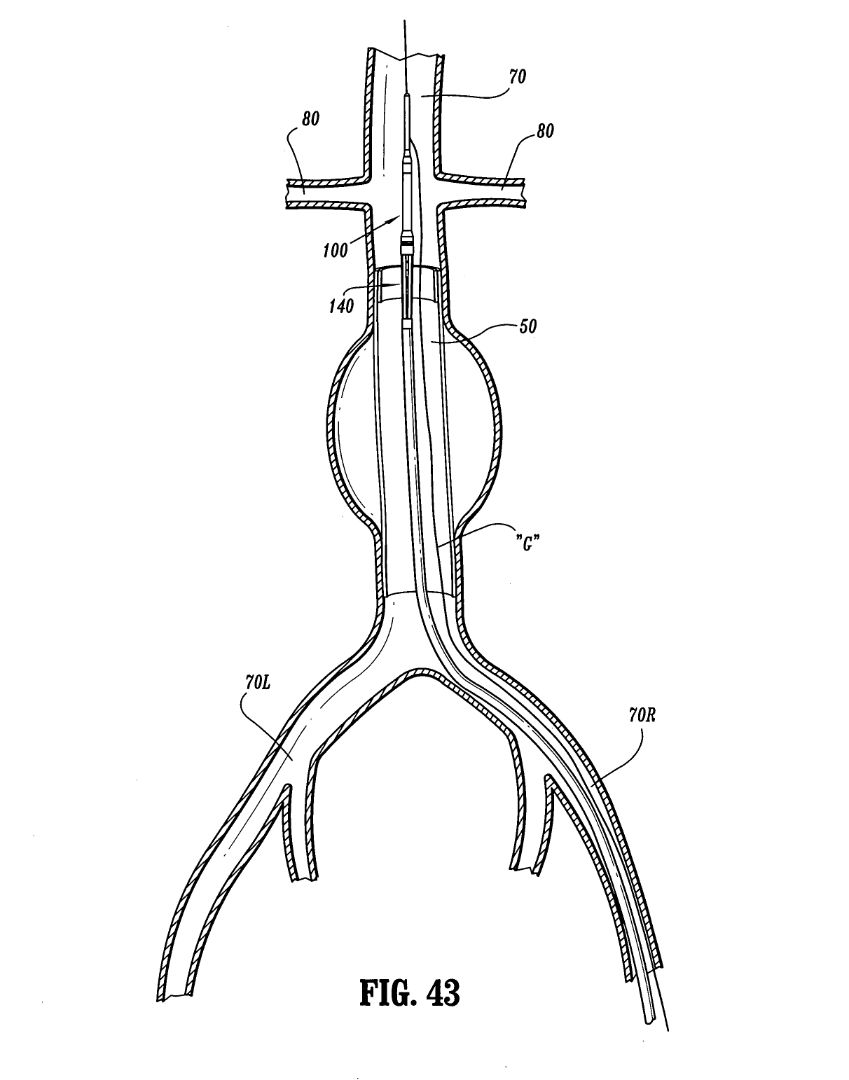

With reference to FIGS. 1, 2, 6, 9, 35 and 36, cap 102 includes an elongate body portion 102a, an atraumatic distal end 102b and a proximal end portion 102c including connecting means 102d (e.g., threads, bayonet-like connecting elements, etc.) for connecting cap 102 to fastener cartridge assembly 150. As seen in FIGS. 35-36, cap 102 further includes a lumen 102e including a first portion 102f axially aligned with and extending distally from distal end 102b and a second portion 102g extending transversely through body portion 102a, e.g., oriented at an angle toward proximal end portion 102c. In use, a guide wire “G” (see FIGS. 35 and 43) may be fed through first and second portions 102f, 102g of lumen 102e and used to guide endovascular fastener 100 through arteries 70L or 70R and/or aorta 70 to aneurysm 60.

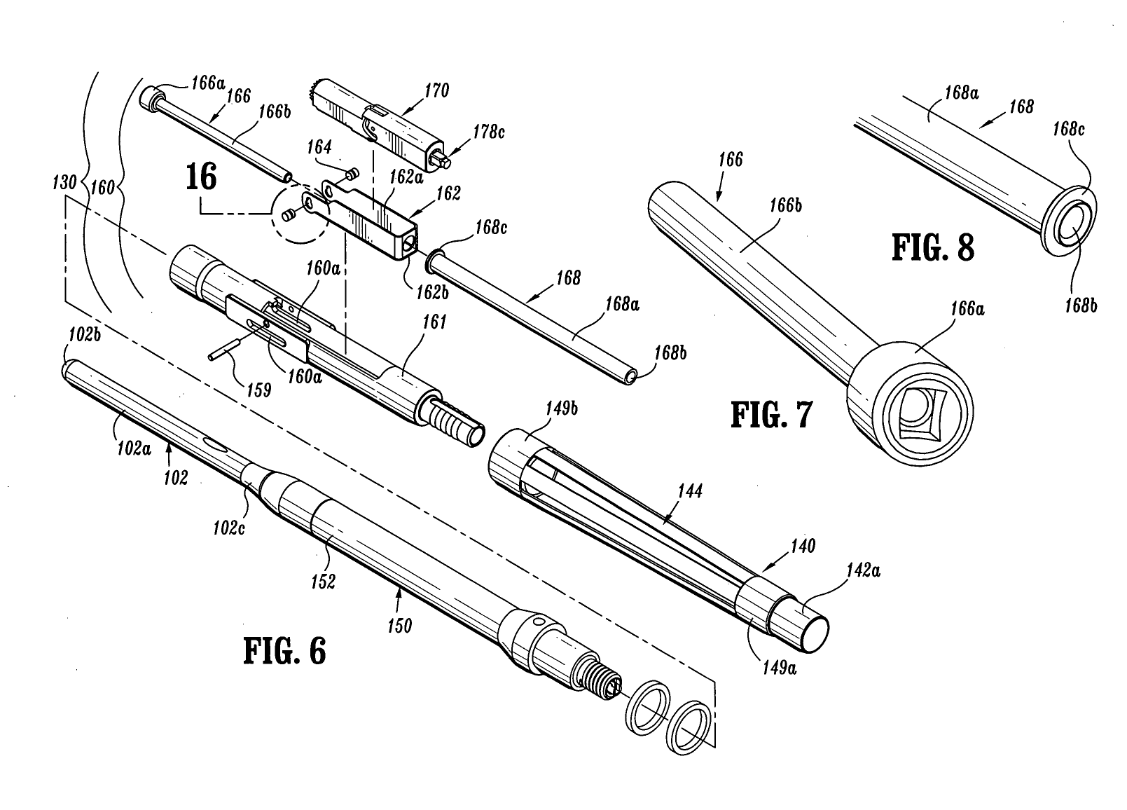

With reference to FIGS. 6 and 9-11, fastener cartridge assembly 150 includes a body portion 152 having a distal end portion 152a configured to engage connecting means 102d of cap 102, and a proximal end portion 152b configured to engage the distal end of drive assembly 130 and expandable portion 140. Fastener cartridge assembly 150 further includes a central shaft 154 rotatably disposed within body portion 152. Central shaft 154 defines an elongate slot 154a configured to receive a penetration limit end of tang 92 of helical fastener 90 as seen in FIG. 10. Central shaft 154 further includes a recess 154b formed at a proximal end thereof (see FIG. 10). Recess 154b is configured to receive a distal end portion 163a of drive shaft 163, as seen in FIGS. 47 and 48, as will be described in greater detail below.

Fastener cartridge assembly 150 further includes a coil 156 disposed between body portion 152 and central shaft 154. Coil 156 operatively engages helical fasteners 90 such that rotation of central shaft 154 results in axial displacement of helical fasteners 90 in a proximal or a distal direction. Accordingly, as central shaft 154 is rotated helical fasteners 90 are deployed from fastener cartridge assembly 150.

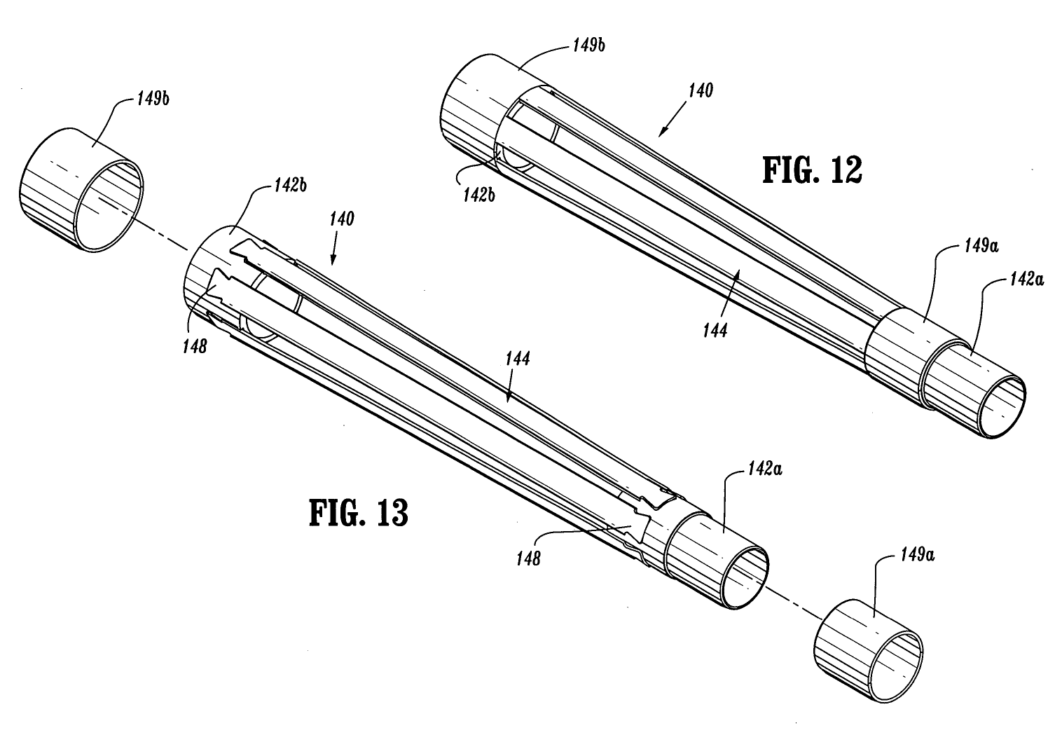

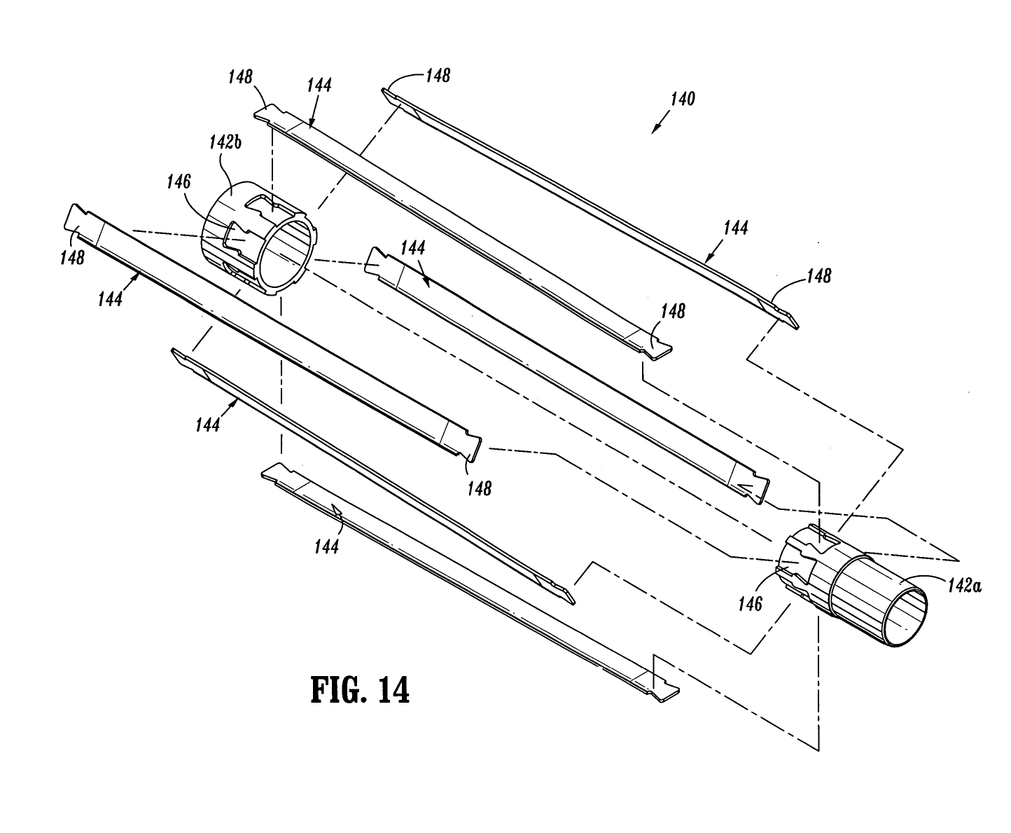

With reference to FIGS. 1, 2, 6 and 12-14, expandable portion 140 includes a proximal hub 142a and a distal hub 142b (FIG. 13) interconnected by a plurality of support members 144 extending therebetween. Each hub 142a, 142b includes a series of grooves 146 formed radially therearound for engaging and/or receiving a tongue 148 formed at each end of support members 144. Expandable portion 140 further includes a proximal locking collar 149a and a distal locking collar 149b. Locking collars 149a, 149b are each configured and dimensioned to surround a respective hub 142a, 142b and maintain support members 144 in operative engagement therewith.

With reference to FIGS. 6 and 15-28, drive assembly 130 includes a yoke assembly 160 (see FIGS. 6 and 15-20) and an applicator head assembly 170 (see FIGS. 6 and 21-28) operatively supported on yoke assembly 160. Yoke assembly 160 is operatively engaged with fastener cartridge assembly 150 and operatively disposed within expandable portion 140. Applicator head assembly 170 is pivotally supported within yoke assembly 160 via a pivot pin 159 extending through body portion 161 of yoke assembly 160 and housing 172 of applicator head assembly 170.

Drive assembly 130 is capable of rotational movement relative to expandable portion 140 while applicator head assembly 170 is capable of pivotal movement between two extreme positions, a first extreme position in which applicator head assembly 170 is coaxially aligned along a longitudinal axis of yoke assembly 160 to load a helical fastener 90 thereon (see for instance FIG. 58), and a second extreme position in which applicator head assembly 170 is perpendicular to the longitudinal axis of yoke assembly 160 (see for instance FIG. 60) to deploy a helical fastener 90 therefrom.

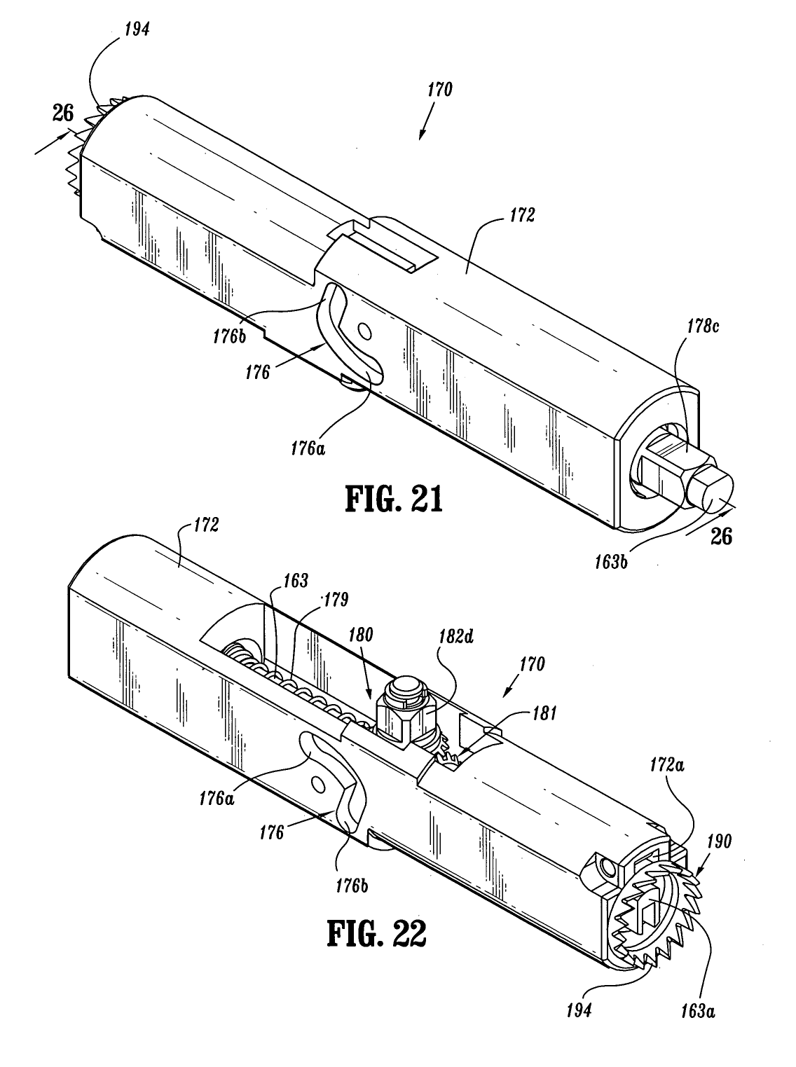

Yoke assembly 160 includes a body portion 161 having a pair of elongate slots 160a formed on either side thereof. Elongate slots 160a extend in a longitudinal direction with respect to body portion 161. Pivot pin 159 extends through body portion 161 at a location adjacent to elongate slots 160a. Yoke assembly 160 further includes a clevis 162 operatively disposed within body portion 161. Clevis 162 includes a pair of arms 162a each having a cam pin 164 extending therethrough. A first half-portion 164a of each cam pin 164 is configured to slidably engage a respective elongate slot 160a of body portion 161. A second half-portion 164b of each cam pin 164 is configured to slidably engage a cam slot 176 (see FIGS. 21, 22 and 27) formed in applicator head assembly 170, as will be described in greater detail below.

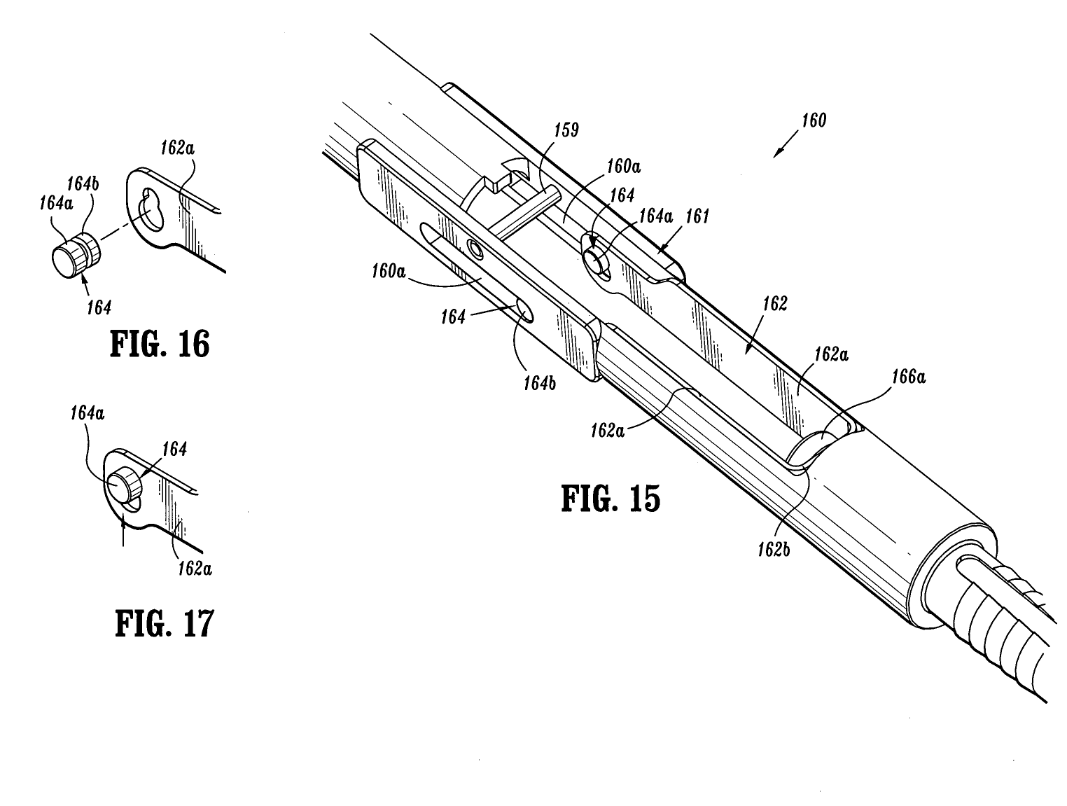

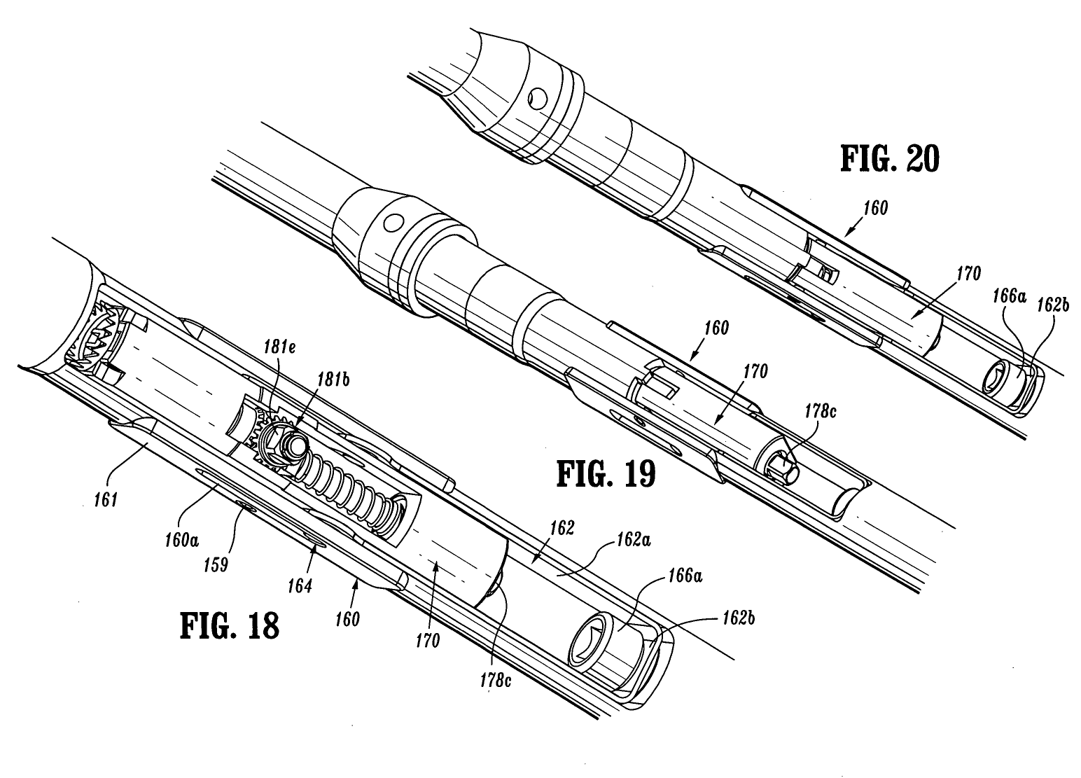

As seen in FIGS. 6, 7, 15, 18-20, 37 and 38, yoke assembly 160 further includes a driver 166 including a coupling member 166a configured to selectively engage a shaped proximal end 178c (see FIGS. 19, 21 and 23-27) of drive screw 178 and a shaped end 182d (see FIGS. 18 and 24-27) of second bevel gear 182. Driver 166 further includes an elongate body portion 166b connected to coupling member 166a and extending through an aperture formed in base portion 162b, between arms 162a, of clevis 162 and into a lumen 168b of pusher 168 (see FIG. 6), as will be described below.

As seen in FIGS. 6, 8, 37 and 38, yoke assembly 160 further includes a pusher 168 including a body portion 168a defining a lumen 168b therethrough, and a flange 168c formed about a distal end of body portion 168a. As seen in FIG. 6, the distal end of body portion 168a of pusher 168 operatively engages the aperture formed in base portion 162b of clevis 162 such that flange 168c engages base portion 162b of clevis 162. As such, lumen 168b is aligned with the aperture of clevis 162. Lumen 168b is configured to slidably and rotatably receive body portion 166b of driver 166 therein.

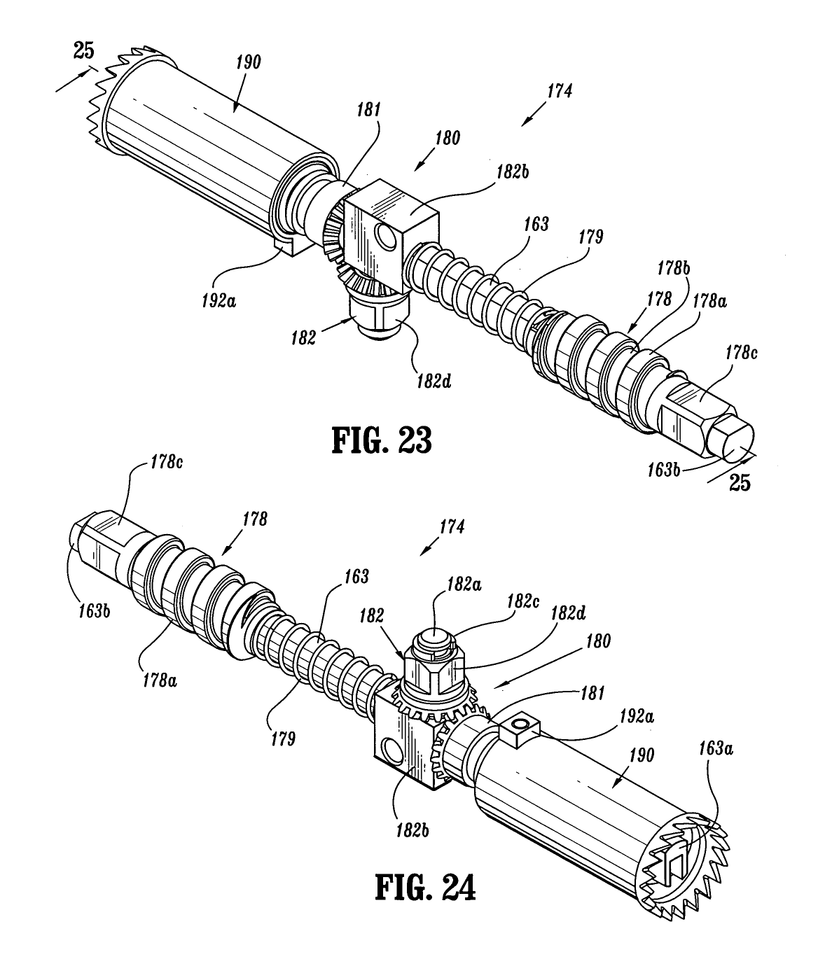

In an embodiment, as seen in FIGS. 6 and 18-28, applicator head assembly 170 includes a housing 172 (see FIGS. 21 and 22) and a drive train 174 (see FIGS. 23 and 24) operatively disposed within housing 172. Housing 172 includes a cam slot 176 (see FIGS. 21 and 22) formed on either side thereof. As seen in FIGS. 21 and 22, each cam slot 176 includes a first portion 176a substantially aligned with a longitudinal axis of housing 172, and a second portion 176b substantially perpendicular to the longitudinal axis of housing 172. Each of cam slots 176 is configured and dimensioned to slidably receive a respective second-half portion 164b of cam pins 164 therein (see for instance FIG. 6).

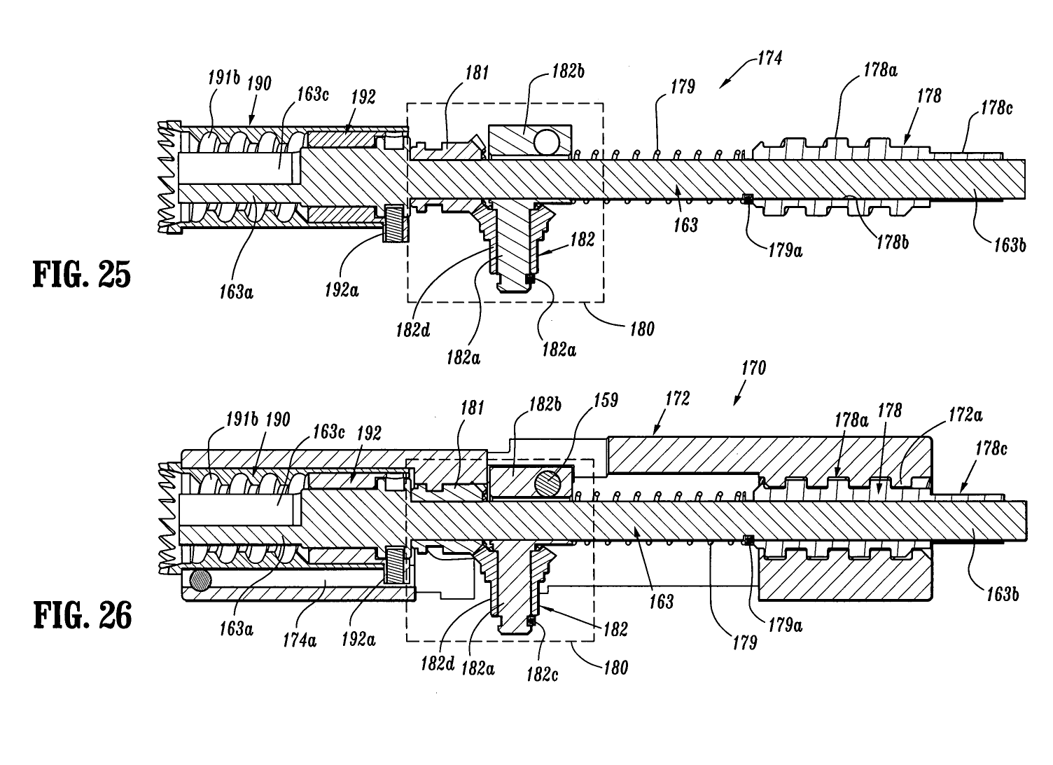

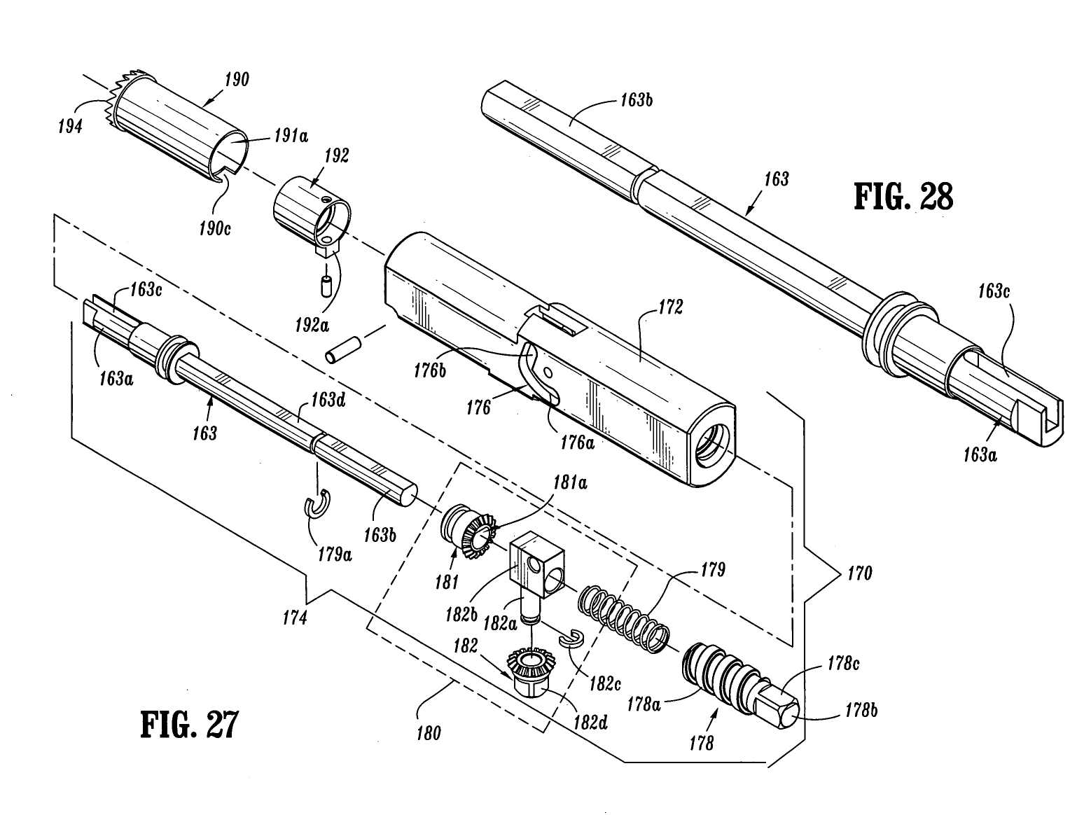

Turning again to FIGS. 23 and 24, drive train 174 includes a drive shaft 163 having a distal end portion 163a and a proximal end portion 163b. Distal end portion 163a is provided with a slot 163c (see FIG. 27) extending therefrom and configured to receive penetration limit end of tang 92 of a helical fastener 90 therein (not shown). Drive shaft 163 has a non-circular transverse cross-section defined by a flattened region 163d extending substantially along a length thereof. Drive train 174 further includes a drive screw 178 operatively attached to, e.g., slidably disposed on, proximal end portion 163b of drive shaft 163. Turning to FIG. 25, drive screw 178 includes a helical thread 178a, a passage 178b (see FIG. 27) extending therethrough for receiving proximal end portion 163b of drive shaft 163 therein, and a shaped proximal end 178c configured to selectively engage coupling member 166a (see for instance FIG. 7) of driver 166, as will be discussed in greater detail below. Helical thread 178a engages threads 172a formed in housing 172, as seen in FIG. 26.

As best seen in FIGS. 22-27, drive train 174 further includes a beveled gear system 180 operatively associated herewith. As seen in FIG. 22, beveled gear system 180 includes a first bevel gear 181 co-axially aligned with and slidably supported on (e.g., keyed, pined, adhered, screwed, etc.) drive shaft 163, and a second bevel gear 182, orthogonally oriented with respect to drive shaft 163 and operatively engaged with first bevel gear 181. As seen in FIG. 27, first bevel gear 181 preferably includes a shaped central lumen 181a configured to engage flattened portion 163d of drive shaft 163. In this manner, as drive shaft 163 or first bevel gear 181 is rotated the other of drive shaft 163 and first bevel gear 181 is also rotated. Second bevel gear 182 is rotatably supported on a stub 182a extending from a gear support box 182b which is rotatably and slidably supported on drive shaft 163. A C-clamp 182c may be used to maintain second bevel gear 182 in position on stub 182a.

In operation, as drive shaft 163 is rotated first bevel gear 181 is rotated about the longitudinal axis which in turn causes second bevel gear 182 to rotate about stub 182a (e.g., an axis orthogonal to the longitudinal axis). Likewise, as second bevel gear 182 is rotated about stub 182a, first bevel gear 181 is rotated to rotate drive shaft 163. Second bevel gear 182 includes a shaped end 182d configured to selectively engage coupling member 166a (see FIG. 7) of driver 166, as will be discussed in greater detail below.

Applicator head assembly 170 further includes an ejection head 190 operatively positioned on distal end portion 163a of drive shaft 163. Ejection head 190 includes a central lumen 191a (see FIG. 27) defining a helical thread 191b (see FIGS. 25 and 26) formed at a distal end thereof. Helical thread 191b is configured and dimensioned to operatively receive a fastener 90.

Applicator head assembly 170 further includes an ejection head stop 192 operatively positioned on, e.g., rotatably supported on, distal end portion 163a of drive shat 163. Ejection head stop 192 includes a radially oriented key 192a configured to engage a corresponding recess 190c formed in a proximal end of ejection head 190 and a slot 172a (see FIG. 22) formed in housing 172 of applicator head assembly 170. In operation, as drive shaft 163 is rotated, ejection head stop 192 and ejection head 190 are prevented from rotating as a result of the engagement of key 192a with slot 172a of housing 172. Ejection head 190 may include a saw-toothed distal end face 194 for engaging and griping aortic graft 50.

Applicator head assembly 170 further includes a coil spring 179 supported on drive shaft 163 between drive screw 178 and gear support box 182b. A C-clamp 179a may be provided about drive shaft 163 between spring 179 and drive screw 178. In this manner, drive shaft 163 is spring biased by coil spring 179 to a proximal-most position as seen in FIGS. 23-26. The axial location of gear support box 182b relative to housing 172 is fixed due to a pivot pin 159 (see FIG. 26) extending through housing 172 and gear support box 182b. In this manner, drive shaft 163 is axially translatable relative to housing 172, as will be described in greater detail below.

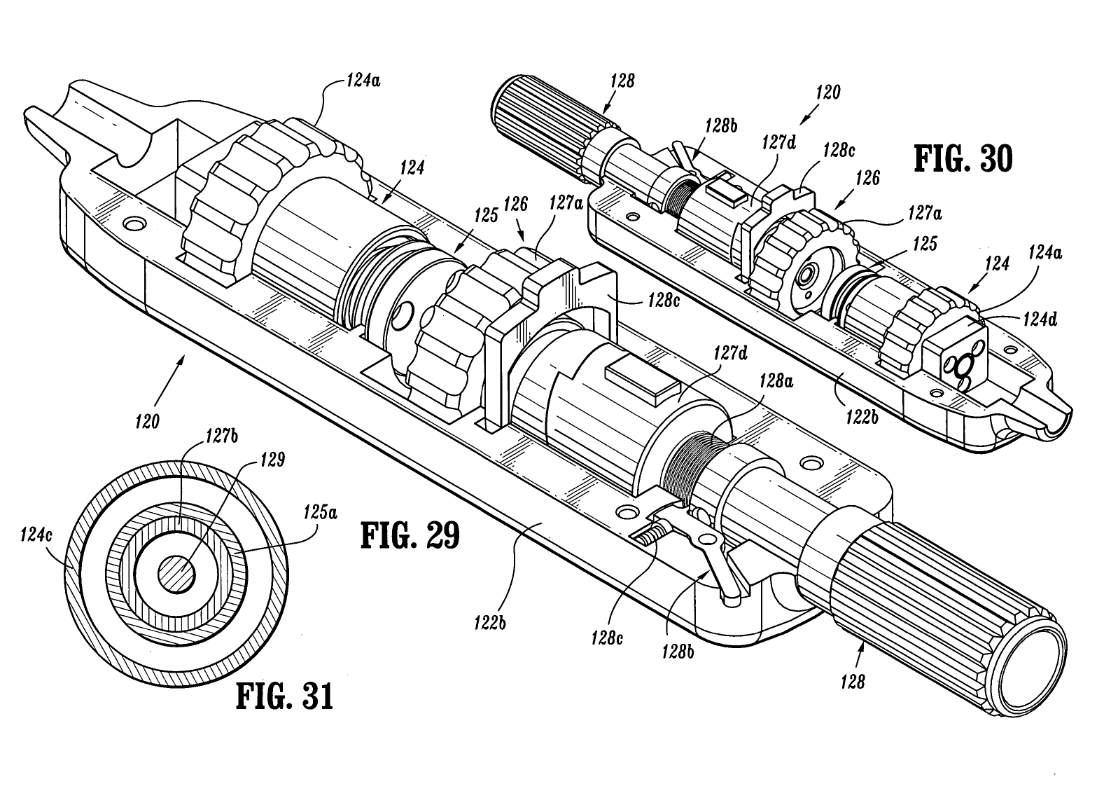

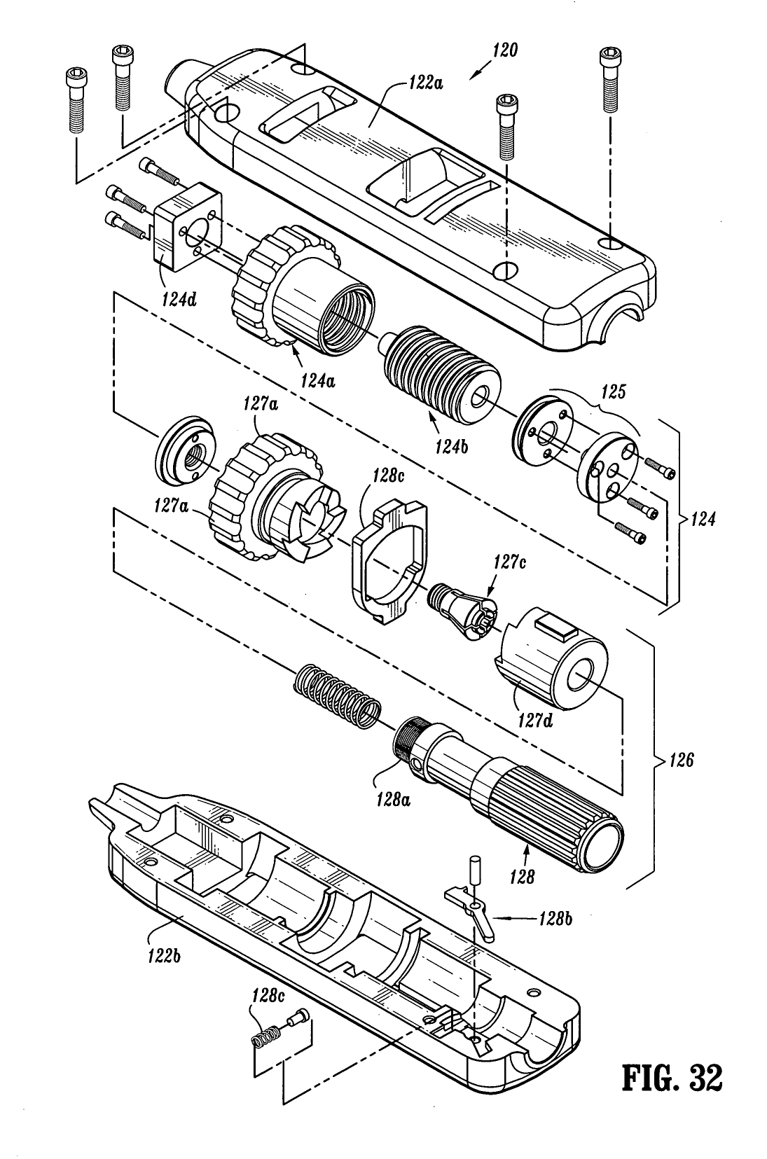

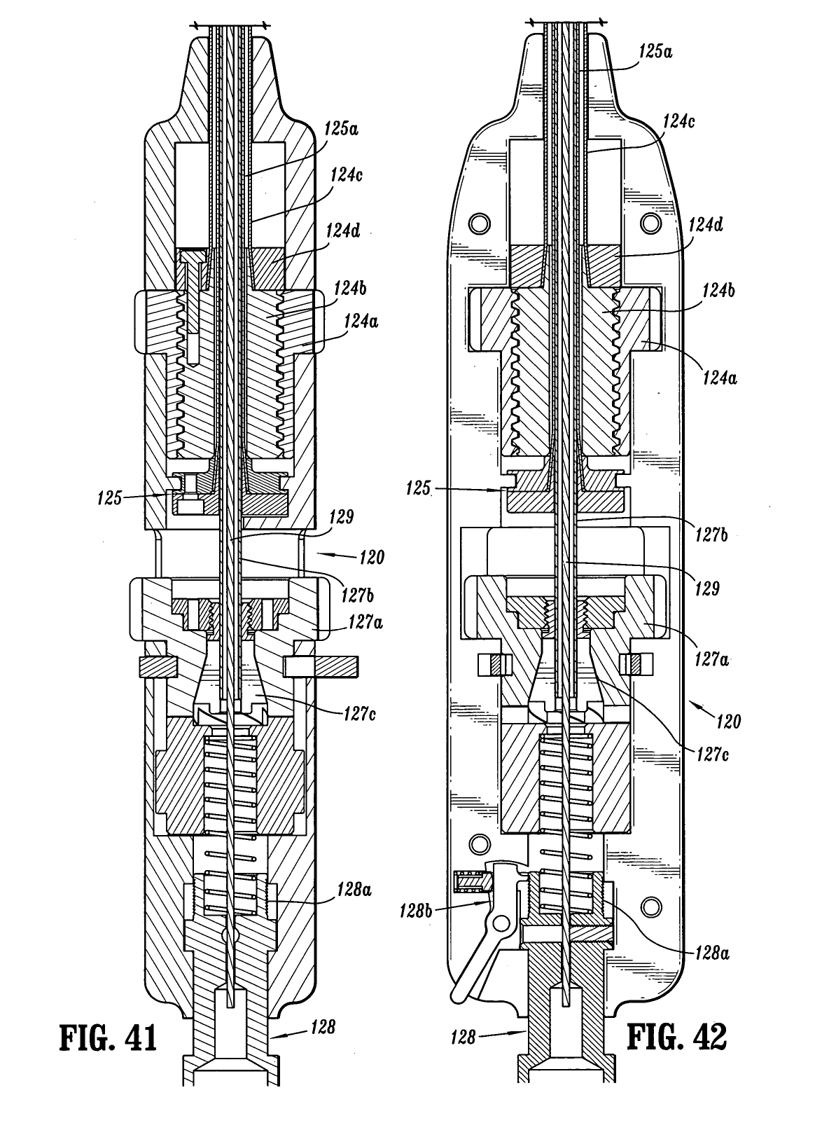

With reference to FIGS. 1, 2, 29-32, 41 and 42, control assembly 120 includes a housing 122 having a pair of half-sections 122a, 122b, configured and adapted to support an expandable portion controller 124 and a drive assembly controller 126 (see FIG. 32).

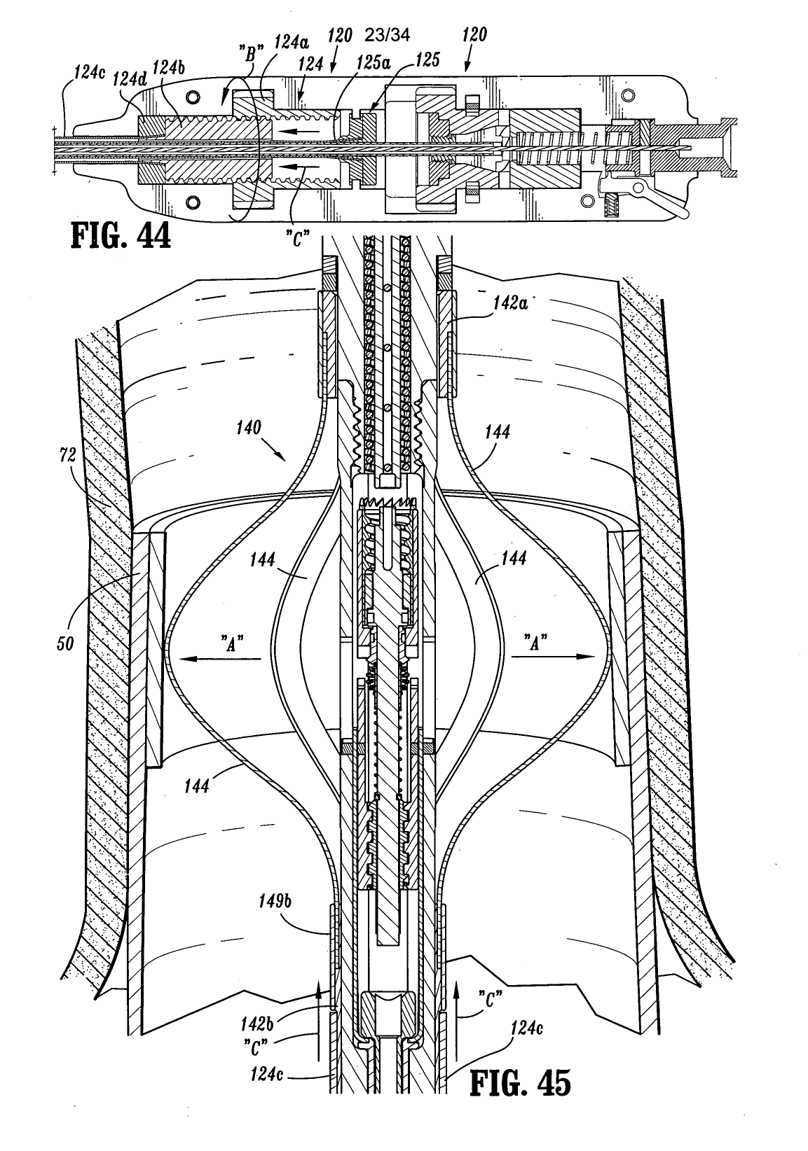

Expandable portion controller 124 controls the expansible force “A” (see FIG. 45) exerted by expandable portion 140 between at least two extreme positions, a first extreme position in which support members 144 of expandable portion 140 are collapsed (i.e., not radially expanded) and a second extreme position in which support members 144 are radially expanded.

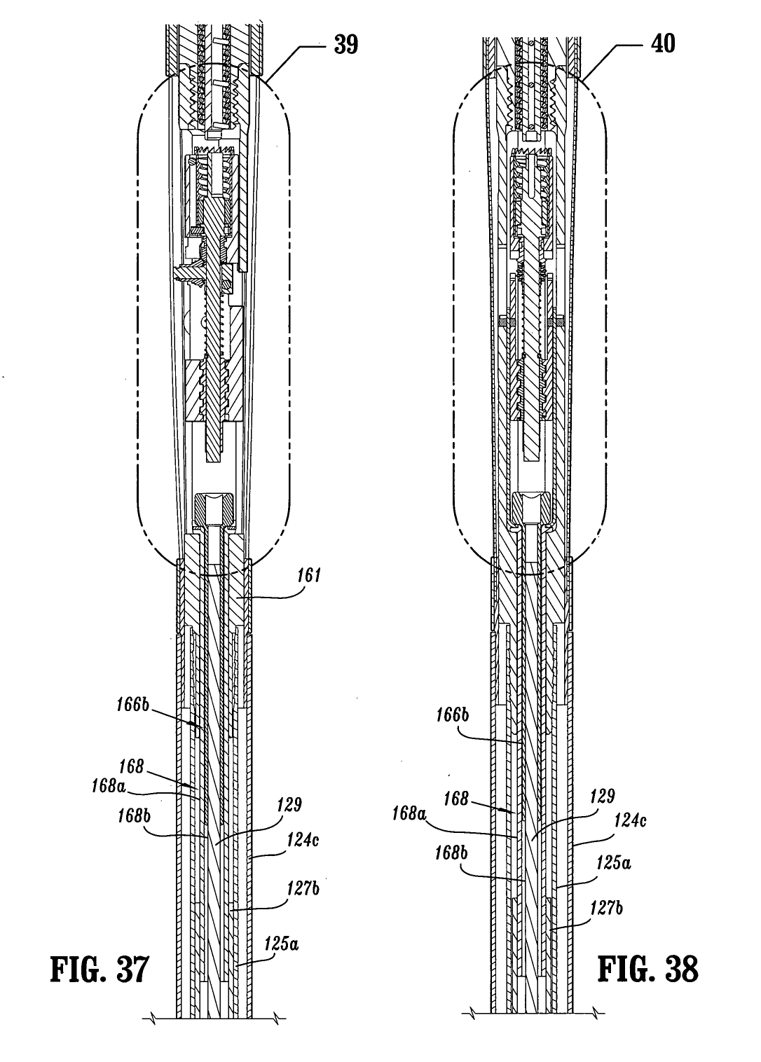

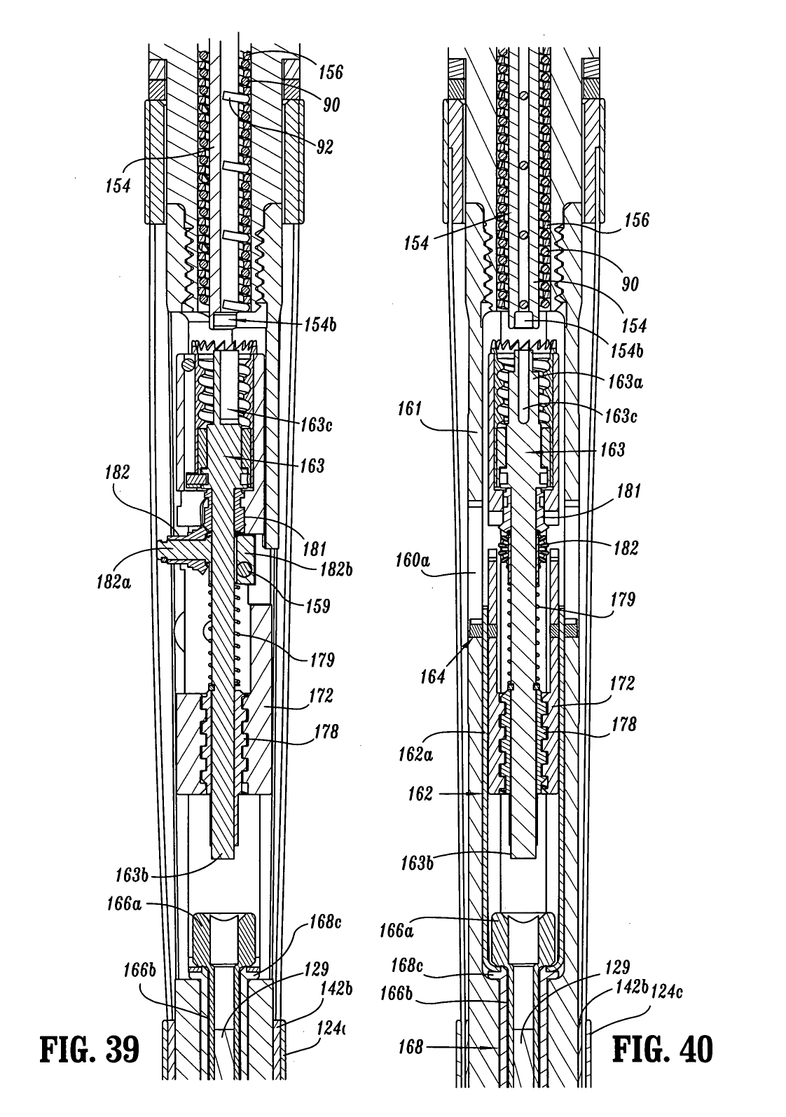

Expandable portion controller 124 includes a knob 124a rotatably supported in housing 122, a spool 124b is in threaded engagement within knob 124a, and an outer tube/sheath 124c (see FIG. 31). Outer sheath 124c has a proximal end fixedly secured between a distal end of spool 124b and a slide block 124d. Slide block 124d is fixedly secured to spool 124b and prevents spool 124b from rotating relative to knob 124a. As seen in FIGS. 37 and 38, a distal end of outer sheath 124c is fixedly secured to proximal hub 142b of expandable portion 140 (see FIGS. 39 and 40). Accordingly, in use, as knob 124a is rotated relative to spool 124b, the threads cause spool 124b to axially translate through knob 124a as slide block 124d prevents spool 124b from rotating relative to housing 122.

Expandable portion controller 124 further includes a flange 125 supported in housing 122 in such a manner that flange 125 is prevented from axial movement relative to handle 122, and a first inner tube 125a (see FIG. 31) is fixedly secured to flange 125 and extends through spool 124b and outer sheath 124c. As seen in FIGS. 37 and 38, a distal end of first inner tube 125a is secured to body portion 161 of yoke assembly 160 which in turn is operatively associated with distal hub 142a of expandable portion 140 (see FIGS. 39 and 40). Accordingly, in use, as will be described in greater detail below, as knob 124a is rotated, spool 124b is displaced distally or proximally, relative to knob 124a, causing outer sheath 124c to displace relative to first inner tube 125a to thereby manipulate expandable portion 140 between the first extreme position in which support members 144 of expandable portion 140 are collapsed (i.e., not radially expanded) and the second extreme position in which support members 144 are radially expanded, as described above.

Drive assembly controller 126 includes a knob 127a rotatably supported in housing 122, and a second inner tube 127b (see FIG. 31), extending through first inner tube 125a, and fixedly secured to knob 127a by a collet 127c (see FIG. 32). As seen in FIGS. 37 and 38, a distal end of second inner tube 127b is operatively connected to body portion 168a of pusher 168. Accordingly, in use, as will be described in greater detail below, as knob 127a is rotated, applicator head assembly 170 (see FIG. 27) is manipulated between the two extreme positions, as described above.

Turning again to FIG. 32, drive assembly controller 126 further includes a drive handle 128 rotatably and translatably supported on housing 122 and a drive shaft 129 (see FIG. 31) fixedly secured to drive handle 128 and extending through second inner tube 127b and lumen 168b of body portion 168a of pusher 168 (see FIGS. 37 and 38). As seen in FIGS. 37 and 40, a distal end of drive shaft 129 is fixedly secured to body portion 166b of driver 166. Accordingly, in use, as will be described in greater detail below, as drive handle 128 is advanced or retracted, driver 166 is advanced or retracted such that coupling member 166a thereof selectively engages or disengages shaped proximal end 178c of drive screw 178 or shaped end 182d of second bevel gear 182. In addition, when coupling member 166 is engaged with drive screw 178 or second bevel gear 182, rotation of drive handle 128 results in rotation of drive screw 178 or second bevel gear 182. In turn, rotation of drive screw 178 or second bevel gear 182 results in rotation of drive shaft 163.

Drive handle 128 may include a series of annular grooves 128a provided at a distal end thereof. Grooves 128a are configured to engage a lock member 128b which is configured to maintain the axial position of drive handle 128 relative to housing 122 and still allow rotation of drive handle 128. Drive handle 128 may be biased to a proximal-most position by a spring 128c.

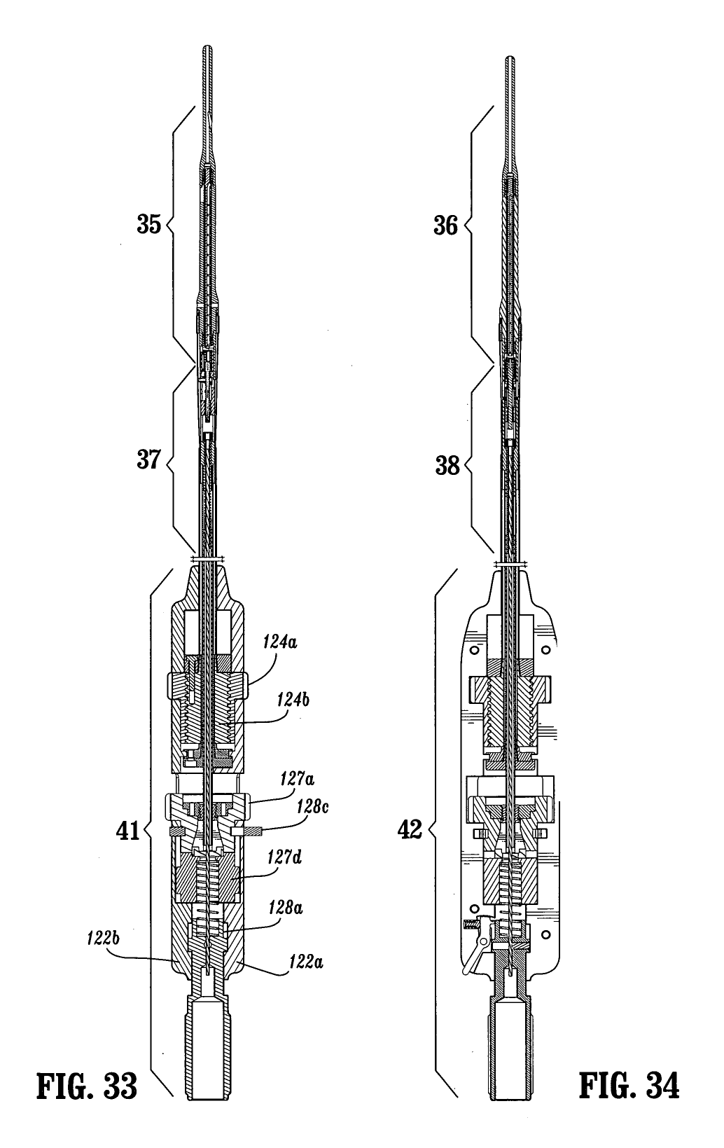

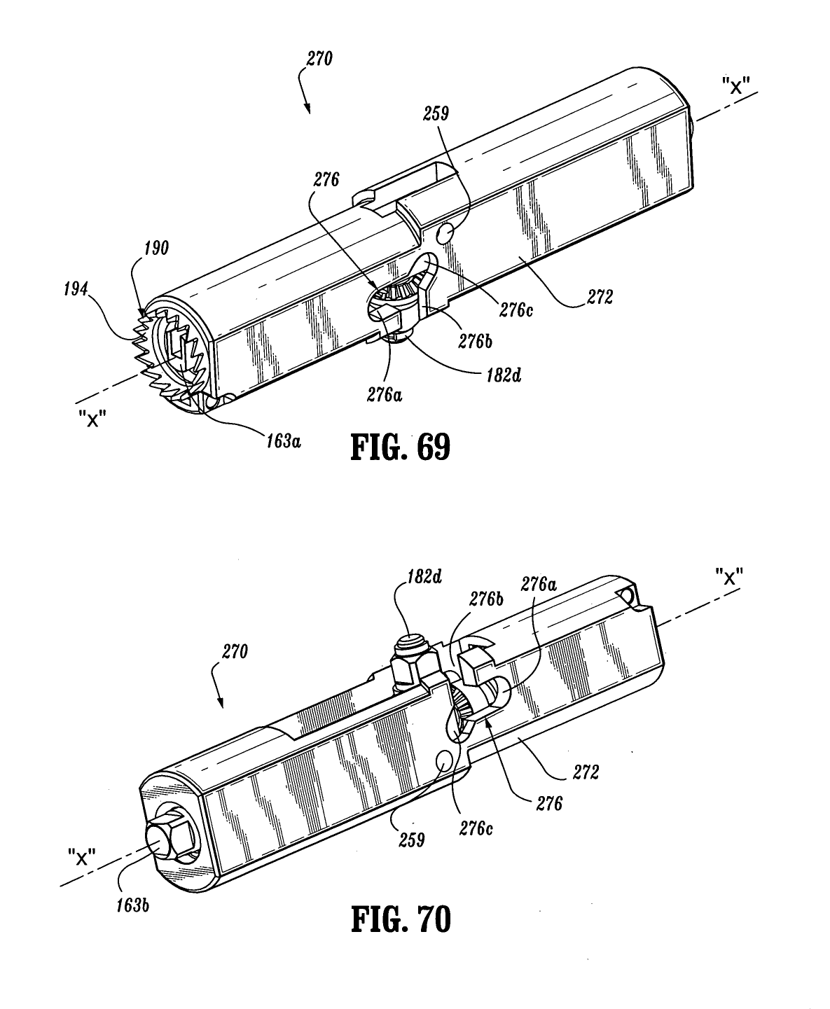

With reference to FIGS. 1-69, and more particularly to FIGS. 33-69, operation of endovascular fastener applicator 100 is shown and described. As seen in FIG. 43, with aortic graft 50 positioned at the aneurysm site, endovascular fastener applicator 100 is fed over and along guide wire “G” until expandable portion 140 of delivery assembly 110 is positioned within aortic graft 50.

As seen in FIGS. 44 and 45, control assembly 120 is then manipulated to radially expand expandable portion 140, in the direction of arrows “A”, from the first extreme position to the second extreme position (i.e., an expanded position). In particular, knob 124a of control assembly 120 is rotated, in the direction of arrow “B”, thus causing expandable portion 140 to radially expand. As knob 124a is rotated, in the direction of arrow “B” (see FIG. 44), spool 124b is moved in a distal direction, as indicated by arrows “C” (see FIG. 44). Since flange 125 is fixed relative to spool 124b, as spool 124b is advanced in direction “C”, relative to first inner tube 125a (see FIGS. 31 and 44), proximal hub 142b is advanced in direction “C” relative to distal hub 142a thereby causing support members 144 to radially expand in direction “A”, as seen in FIG. 45. Radial expansion of expandable portion 140 results in the formation of interstitial regions 143 between support members 144 through which helical fasteners 90 may be fired into aortic graft 50. In addition, radial expansion of expandable portion 140 results in support members 144 pressing against the inner surface of aortic graft 50 to thereby maintain aortic graft 50 in position at the site of the aneurysm.

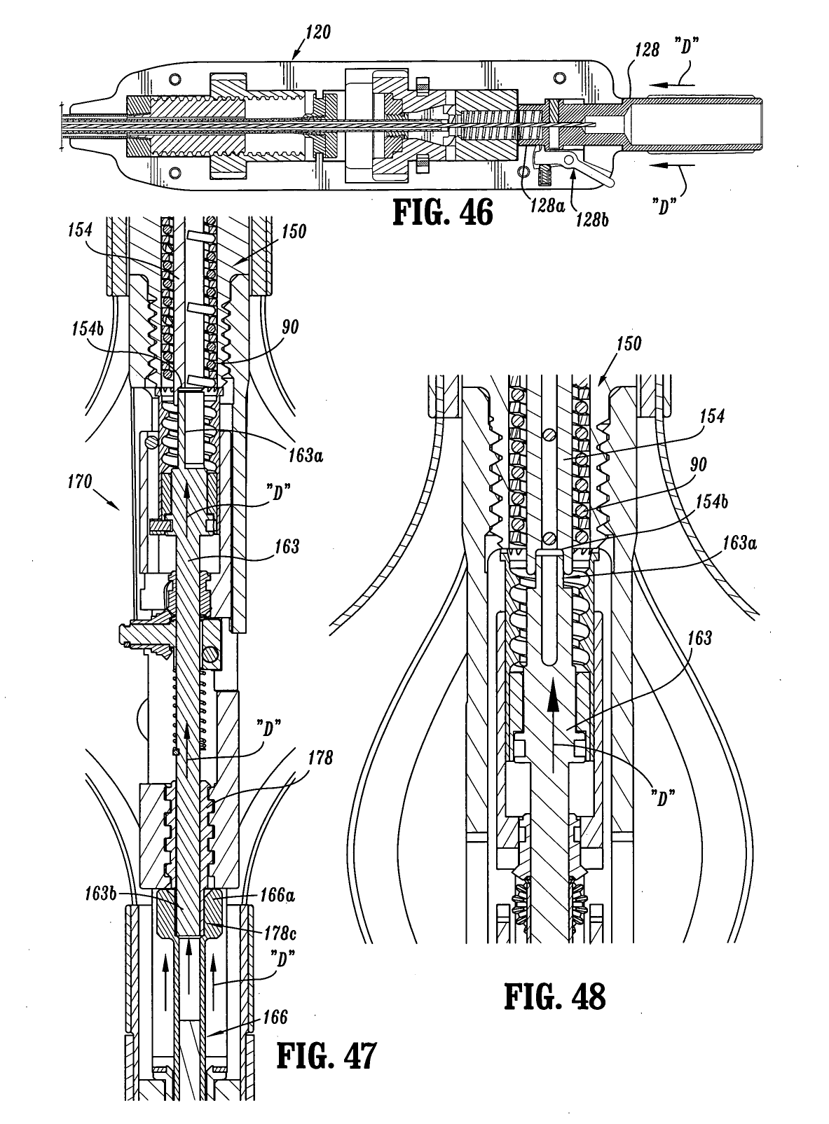

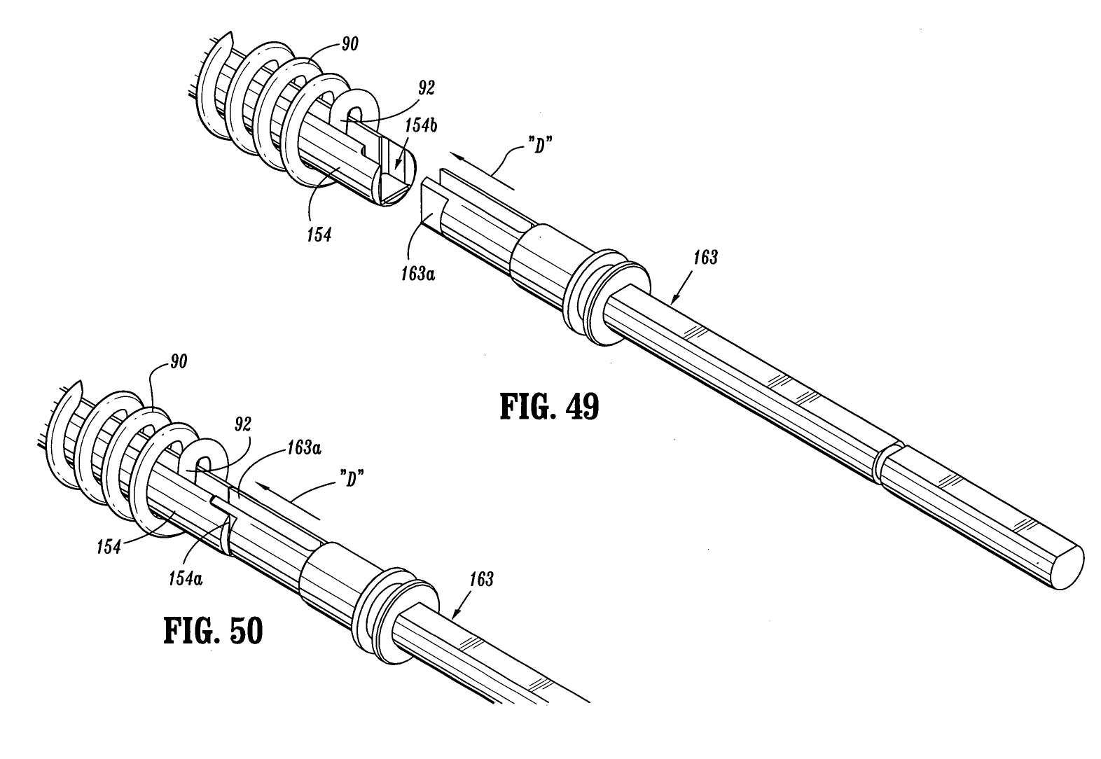

As seen in FIGS. 46-50, loading of a helical fastener 90, retained/stored in fastener cartridge assembly 150, onto applicator head assembly 170, will be shown and described. In particular, drive handle 128 is advanced in a distal direction, as indicated by arrow “D”, until coupling member 166a of driver 166 engages shaped proximal end 178c of drive screw 178. In advancing drive handle 128 in the “D” direction, once proximal end portion 163b of drive shaft 163 bottoms out in coupling member 166a further advancement of drive handle 128 in the “D” direction results in drive shaft 163 also advancing in the “D” direction. As seen in FIGS. 49 and 50, drive handle 128 (see FIG. 46) is advanced in a distal or “D” direction until distal end portion 163a of drive shaft 163 engages (i.e., is coupled with, received in, mated with, etc.) recess 154b formed at the proximal end of central shaft 154 of fastener cartridge assembly 150.

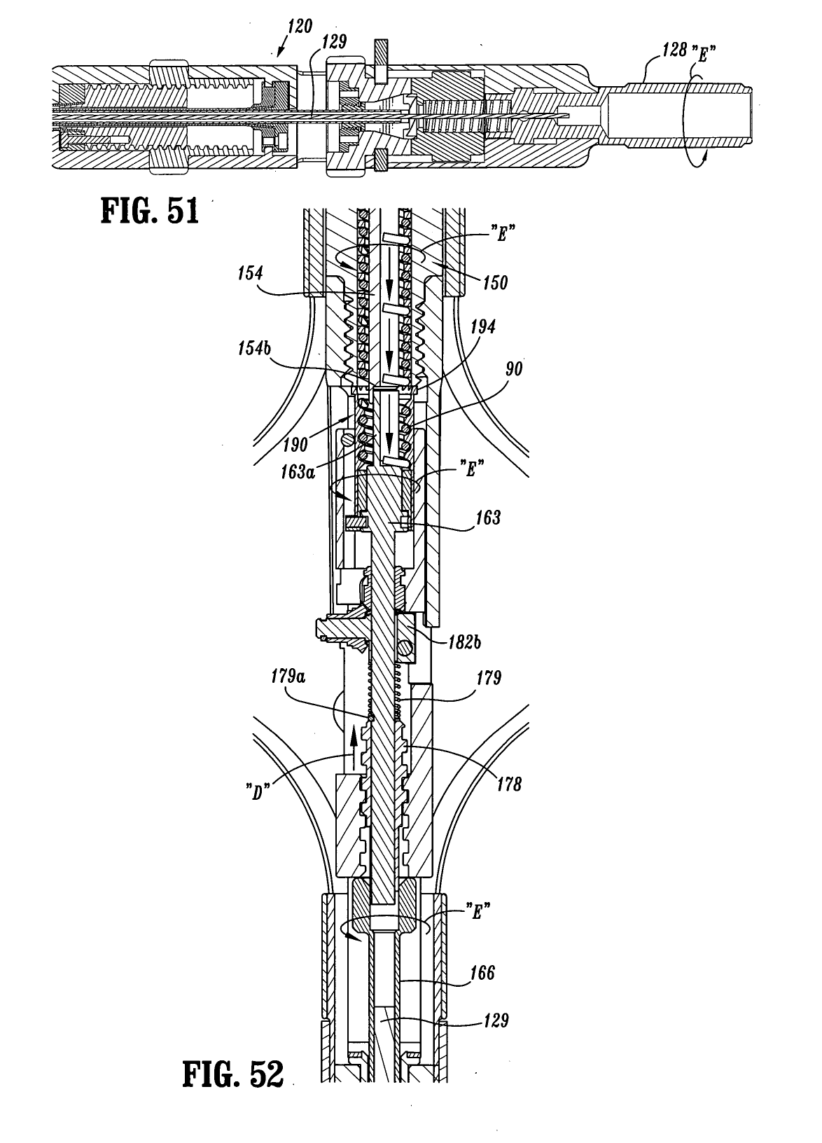

As seen in FIGS. 51 and 52, with distal end portion 163a of drive shaft 163 engaged with recess 154b of central shaft 154, drive handle 128 is rotated in the direction of arrow “E”. Rotation of drive handle 128 in the “E” direction results in the rotation of each of drive shaft 129, driver 166, drive shaft 163 and central shaft 154 in the “E” direction. Drive handle 128 is rotated in the “E” direction until a proximal-most helical fastener 90 is backed-out of fastener cartridge assembly 150 and loaded onto/into ejection head 190 of applicator head assembly 170.

As seen in FIG. 52, rotation of drive handle 128 also causes drive screw 178 to be displaced in the “D” direction and to compress spring 179 against gear support box 182b. Drive screw 178 presses against C-clamp 179a which in turn displaces drive shaft 163 distally. Additionally, ejection head 190 is moved distally such that saw-toothed distal end face 194 extends distally out of housing 172 of applicator head assembly 170.

In addition, as seen in FIG. 46, lock member 128b may be employed to engage grooves 128a of drive handle 128. In this manner, lock member 128b may maintain drive handle 128 in the distally advanced position at least during rotation of drive handle 128 for loading of helical fastener 90 into applicator head assembly 170.

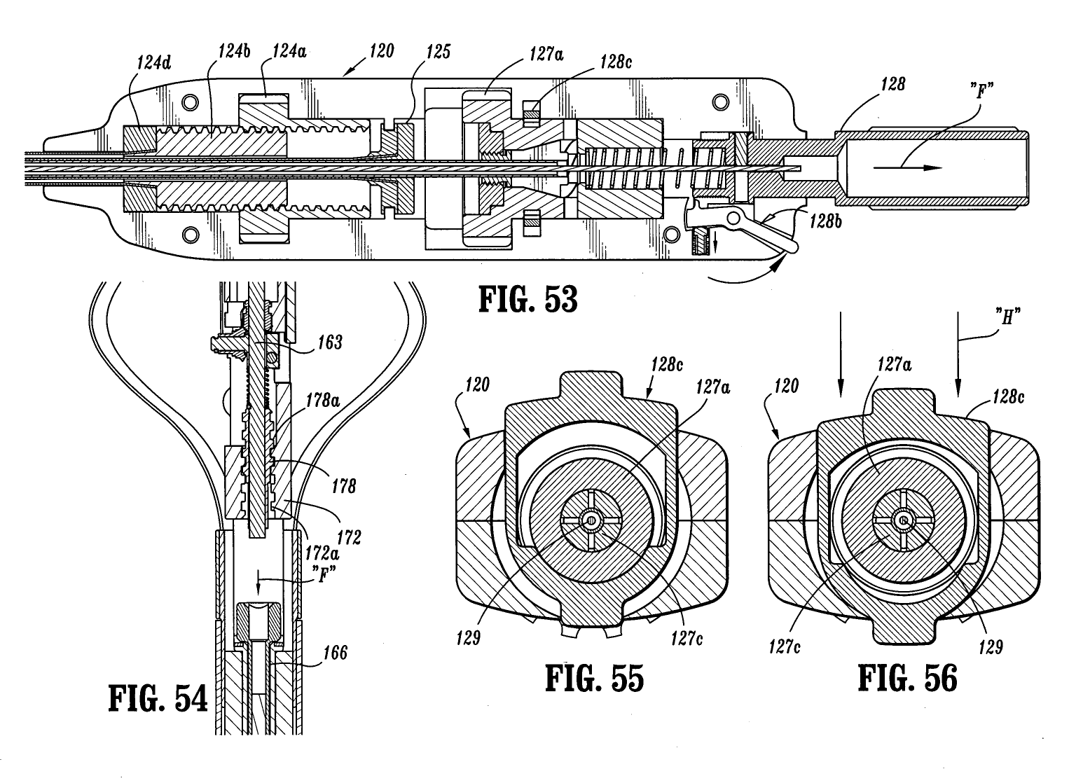

With a helical fastener 90 loaded into applicator head assembly 170, as seen in FIGS. 53 and 54, lock member 128b may be manipulated to disengage from drive handle 128 and enable drive handle 128 to be withdrawn in the direction of arrow “F” to disengage driver 166 from drive screw 178. As seen in FIG. 54, drive shaft 163 remains in the advanced/distal position by the interaction of threads 178a of drive screw 178 with threads 172a of housing 172.

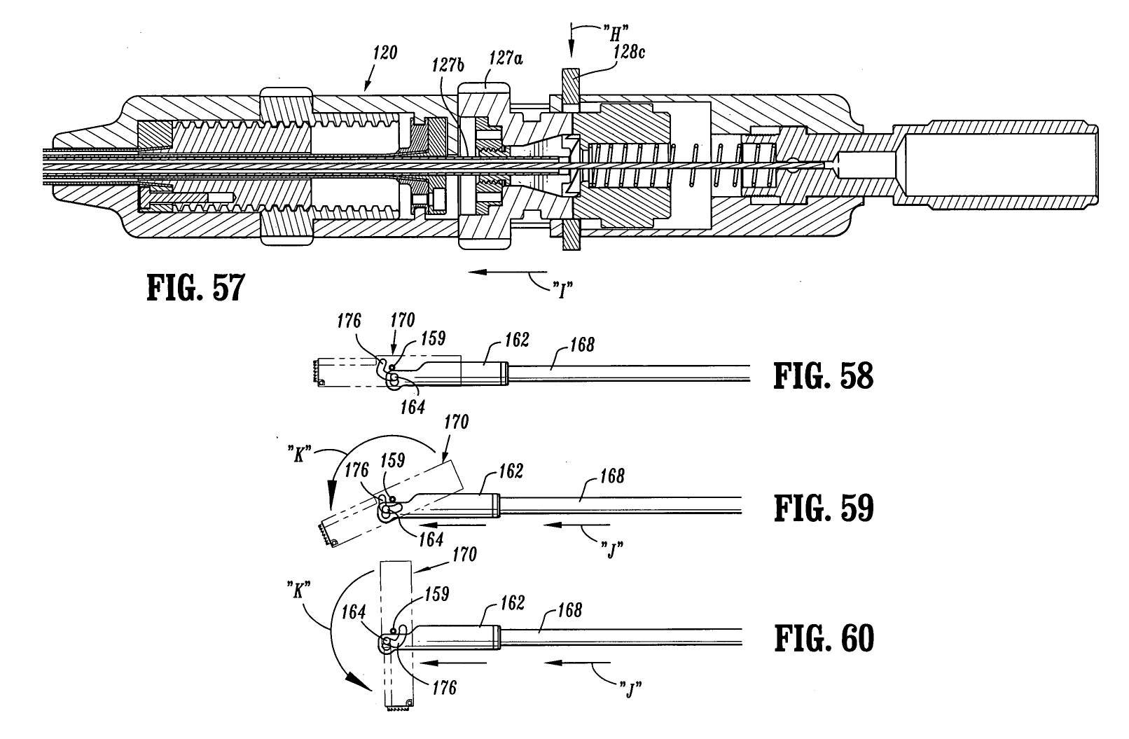

As seen in FIGS. 55-57, a safety/lock-out 128c is moved in the direction of arrow “H” in order to free knob 127a and enable axial displacement of knob 127a in the direction of arrow “I”. With knob 127a free to move in an axial direction, as seen in FIGS. 57-60, knob 127a is displaced in a distal direction, i.e., the “I” direction. As seen in FIGS. 58-60, movement of knob 127a in the “I” direction results in pivoting of applicator head assembly 170 about pivot pin 159, as indicated by arrow “K”, from the first extreme position to the second extreme position, as described above. In particular, as knob 127a is moved in the “I” direction second inner tube 127b advances pusher 168 which in turn advances clevis 162 thereby advancing cam pins 164 through elongate slots 160a (see FIGS. 15 and 40) of body portion 161 and cam slots 176 of applicator head assembly 170. As cam pins 164 travel through elongate slots 160a and cam slots 176, cam pins 164 urge applicator head assembly 170 to approximately 90° from an axially aligned position to an orthogonal position.

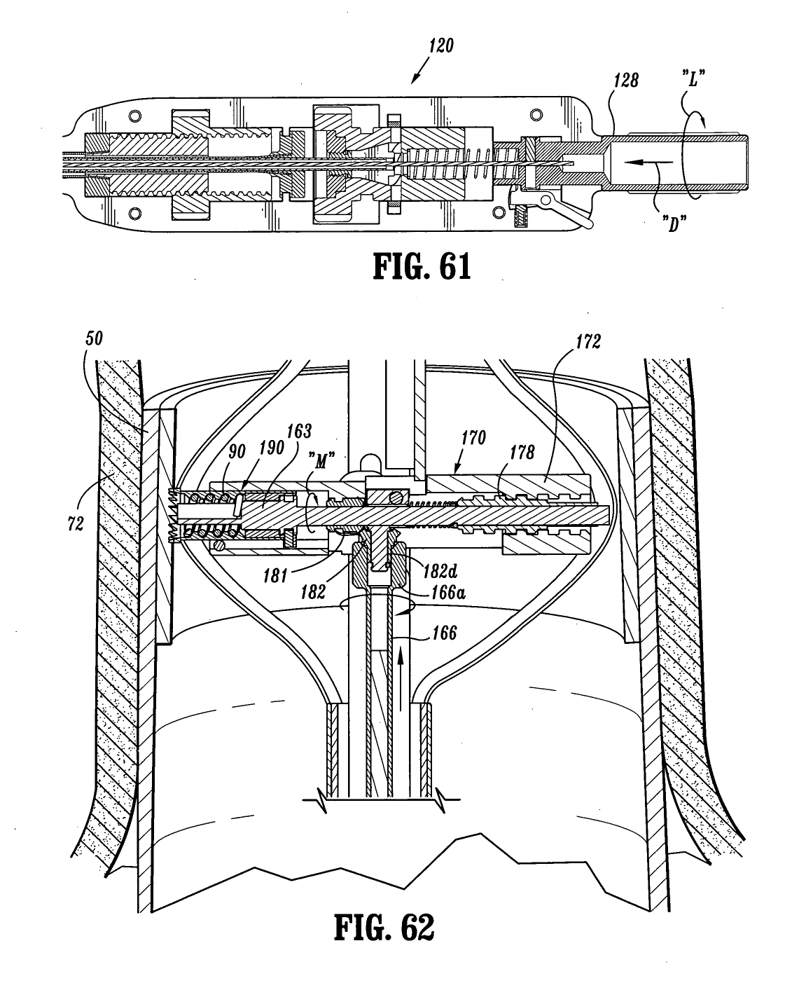

As seen in FIGS. 61-64, with applicator head assembly 170 in the second extreme position (i.e., ejection head 190 of applicator head assembly 170 is oriented toward aortic graft 50), drive handle 128 is once again advanced in the distal or “D” direction such that coupling member 166a of driver 166 engages shaped end 182d of second bevel gear 182. With driver 166 in operative engagement with second bevel gear 182, drive handle 128 is rotated in the direction of arrow “L”. Rotation of drive handle 128 in the “L” direction results in rotation of drive shaft 129, driver 166, and second bevel gear 182 in the “L” direction. Rotation to second bevel gear 182 in the “L” direction transmits rotation, in the direction of arrow “M”, to drive shaft 163 via its inter-engagement with first bevel gear 181.

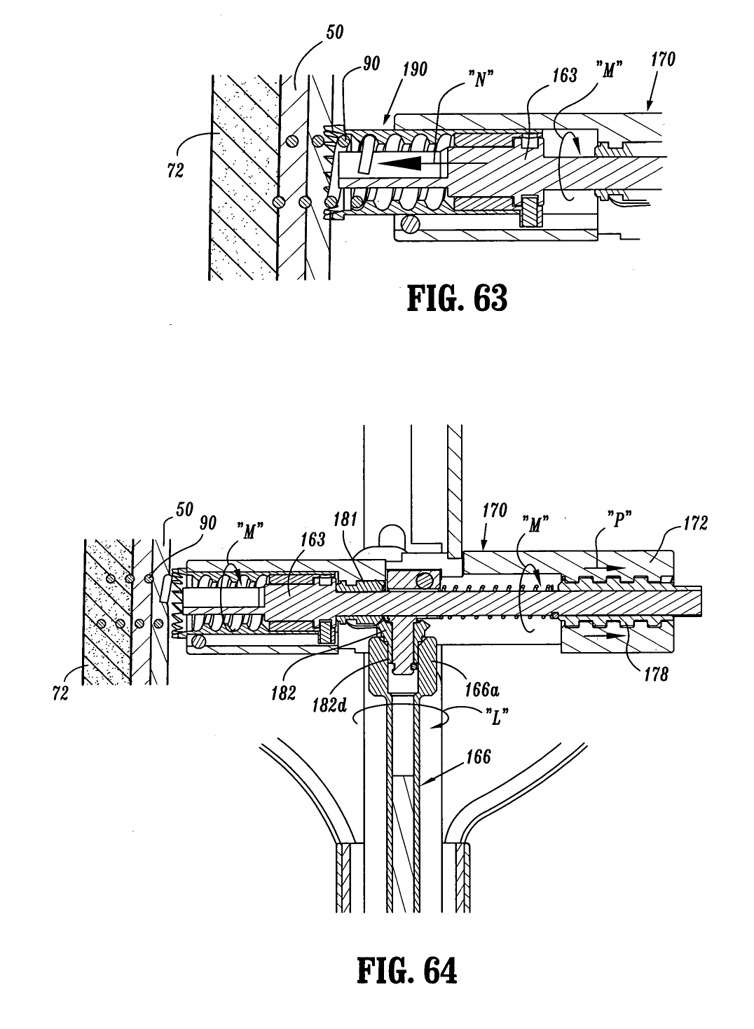

As seen in FIG. 63, as drive handle 128 is rotated in the “L” direction, helical fastener 90, loaded onto applicator head assembly 170, is driven out of ejection head 190 and into aortic graft 50 and aortic wall 72 as indicated by arrow “N”. As seen in FIG. 64, as helical fastener 90 is being driven into aortic graft 50 by rotation of drive shaft 163, drive screw 178 is simultaneously returned to its position in housing 172, as indicated by arrow “P”, thereby withdrawing drive shaft 163 and ejection head 190 from aortic graft 50.

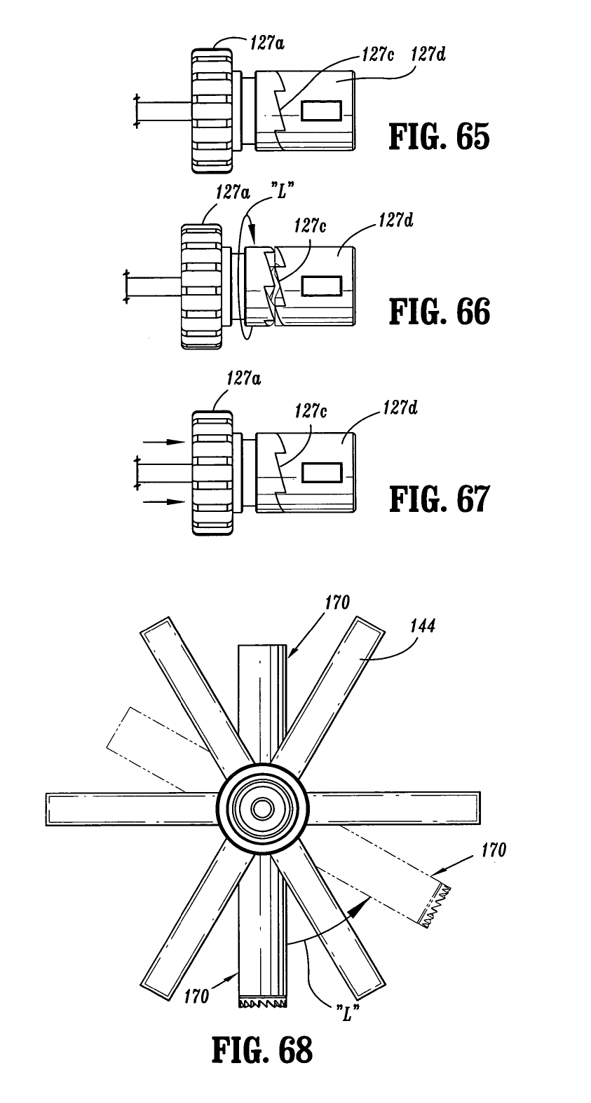

With helical fastener 90 in place, applicator head assembly 170 may be returned to the first extreme condition and re-loaded with another helical fastener 90 retained in fastener cartridge assembly 150 and re-oriented (i.e., rotated to an adjacent interstitial region 143 between support members 144). In particular, as seen in FIGS. 65-68, as knob 127a is rotated in a direction indicated by arrow “L” applicator head assembly 170 is caused to be rotated. It is envisioned that a rotation limiter 127d, including camming surfaces 127e, may be provided which indicates to the user when applicator head assembly 170 has been rotated to an adjacent interstitial region 143 for application of another helical fastener. Preferably, as many helical fasteners 90 may be deployed as are necessary to adequately fasten aortic graft 50 to aortic wall 72.

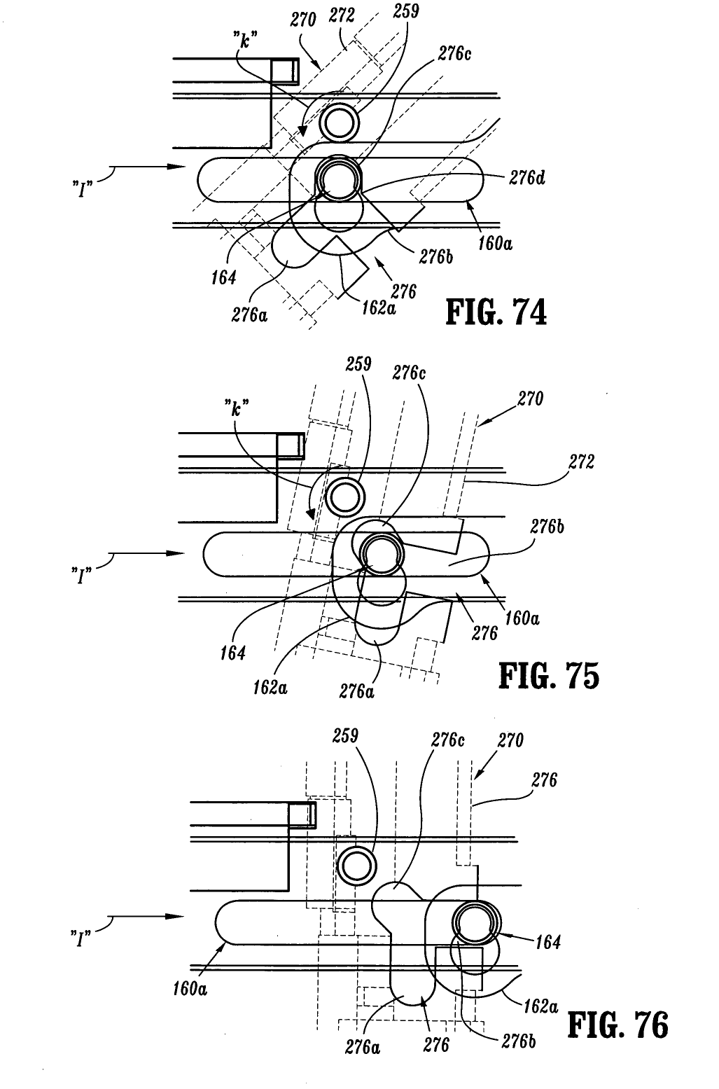

Turning now to FIGS. 69-76, an applicator head assembly, in accordance with an alternate embodiment of the present disclosure, is generally designated as 270. Applicator head assembly 270 is substantially similar to applicator head assembly 170 and will only be described in detail herein to the extent necessary to identify differences in construction and operation.

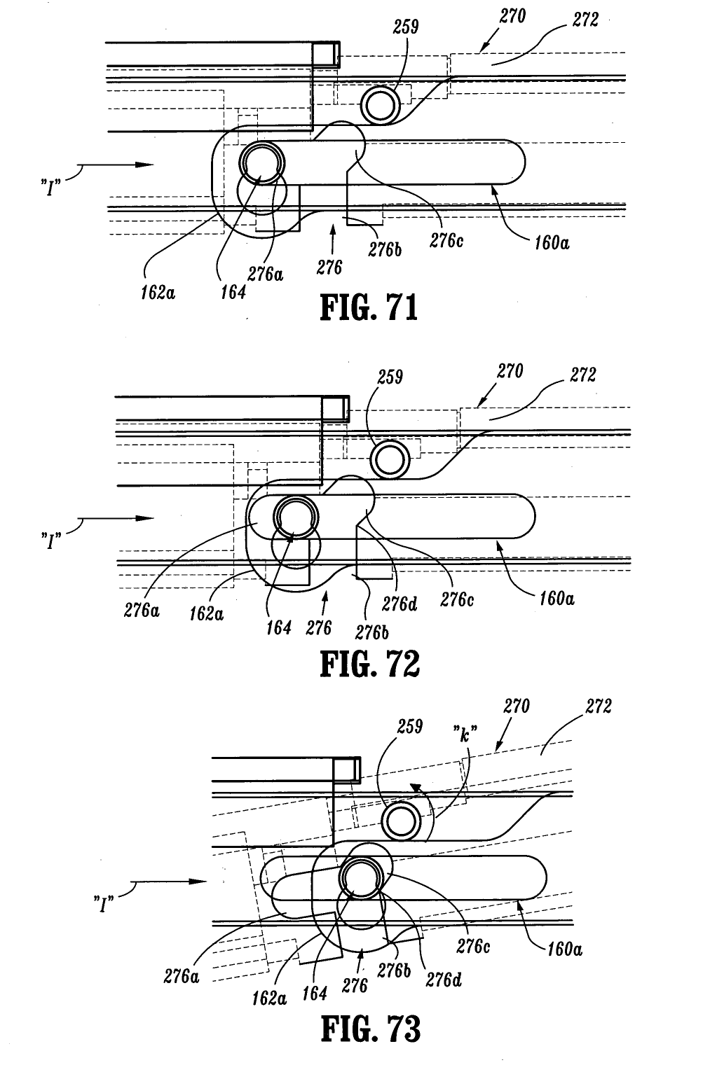

As seen in FIGS. 69-76 and, in particular, FIGS. 69 and 70, applicator head assembly 270 includes a housing 272 defining a cam slot 276 formed in either side thereof. Each cam slot 276 includes a first portion 276a substantially aligned with a central longitudinal “X” axis of housing 272, a second portion 276b extending substantially perpendicularly to the longitudinal “X” axis of housing 272, and a third portion 276c angled with respect to the longitudinal “X” axis of housing 272. Second portion 276b of cam slot 276 extends through a bottom of housing 272. Third portion 276c of cam slot 276 extends at an angle from the intersection of first portion 276a and second portion 276b of cam slot 276. Third portion 276c is angled at about 45° relative to the longitudinal “X” axis. Cam slots 276 are disposed distal of pivot pin 259 (see FIGS. 71-76) when applicator head assembly 270 is in the first extreme position.

Turning now to FIGS. 71-76, the pivoting of applicator head assembly 270 about pivot pin 259, from the first extreme position to the second extreme position, is shown and described. In particular, as seen in FIGS. 71 and 72, as knob 127a (see FIG. 57) is moved in the “I” direction, second inner tube 127b (see FIG. 57) axially moves pusher 168 (see FIGS. 6, 40 and 56-60) which in turn axially moves clevis 162 (see FIGS. 6 and 40), thereby axially moving cam pins 164 (see FIGS. 6 and 40) through elongate slots 160a (see FIGS. 6 and 40) of body portion 161 (see FIGS. 6 and 40) and through first portion 276a of cam slot 276 of applicator head assembly 270. As seen in FIG. 73, cam pins 164 are axially moved through first portion 276a of cam slot 276 until cam pins 164 contact or otherwise operatively engage a shoulder 276d (i.e., the intersection between second portion 276b and third portion 276c of cam slot 276). Upon continued axial movement of cam pins 164 through elongate slots 160a, cam pins 164 enter third portion 276c of cam slots 276 and cause applicator head assembly 270 to begin to rotate about pivot pin 259, as indicated by arrow “K”.

As seen in FIGS. 73-75, cam pins 164 move axially past pivot pin 259. In particular, continued axial movement of cam pins 164 through elongate slots 160a, beyond pivot pin 259, causes applicator head assembly 270 to rotate, about pivot pin 259, to the second extreme position. As seen in FIG. 76, when applicator head assembly 270 has been rotated to the second extreme position, second portion 276b of cam slot 276 is substantially axially aligned with the longitudinal axis of elongate slots 160a. Accordingly, with applicator head assembly 270 in the second extreme position, cam pin 164 may move through second portion 276b of cam slot 276. This process is reversed (i.e., cam pins 164 are moved in a direction opposite to arrow “I”) in order to return applicator head assembly 270 to the first extreme position.

It will be understood that various modifications may be made to the embodiments disclosed herein. For example, while specific preferred embodiments of the endovascular fastener applicator have been described in detail, structures that perform substantially the same function in substantially the same way to achieve substantially the same result may also be used. For example, the expandable portion may include expanding wires for supporting a prosthetic device in contact with a vessel wall. Also the fastener guide may be implanted completely through the thickness of the aortic graft. Further, the helical fasteners may be constructed from various suitable materials or may embody one continuous fastener that is severable at the point of insertion. Therefore, the above description should not be construed as limiting, but merely as exemplifications of preferred embodiments, those skilled in the art will envision other modifications within the scope and spirit of the present disclosure.

1. An endovascular fastener applicator for endoluminally fastening a prosthetic graft to a vessel with at least one fastener comprising:
a delivery assembly configured for positioning within a vessel, the delivery assembly including:
an expandable portion disposed adjacent a distal end of an outer sheath and being expandable to support a prosthetic in contact with an inner surface of a vessel;
a yoke assembly disposed within the expandable portion;
an applicator head assembly pivotably mounted to the yoke assembly and movable between a loading position longitudinally aligned with the yoke assembly, and a firing position oriented off axis to the yoke assembly; and
a fastener assembly positioned at a distal end of the expandable portion, the fastener assembly retaining at least one fastener therein; and
a control assembly mounted to a proximal end of the outer sheath for extracorporeal control of the delivery assembly.

2. The applicator as recited in claim 1, wherein the control assembly has a first knob to expand the expandable portion.

3. The applicator as recited in claim 2, wherein the control assembly includes a second knob to pivot the applicator head assembly from the loading position to the firing position.

4. The applicator as recited in claim 3, wherein the control assembly includes a third knob to rotate the applicator head assembly about a longitudinal axis of the outer sheath.

5. The applicator as recited in claim 4, wherein the second knob of the control assembly withdraws a fastener out of the fastener assembly and drives the fastener into tissue.

6. The applicator as recited in claim 1, wherein the fastener is a helical coil fastener.

7. An endovascular fastener applicator for endoluminally fastening a prosthetic graft to a vessel with at least one fastener comprising:
an outer sheath;
a delivery assembly for delivering a graft to a site within a vessel and for fastening a prosthetic graft to a vessel by passing a fastener therethrough, the delivery assembly including:
an expandable portion disposed adjacent a distal end of the outer sheath and deployable to support a prosthetic in contact with an inner surface of a vessel;
a drive assembly operatively disposed within the expandable portion, the drive assembly including:
a yoke assembly for guiding and supporting an applicator head assembly; and
an applicator head assembly pivotably mounted to the yoke assembly and movable between a loading position longitudinally aligned with the yoke assembly, and a firing position oriented substantially perpendicular to the yoke assembly; and
a fastener assembly positionable at a distal end of the expandable portion, the fastener assembly retaining at least one fastener therein; and
a control assembly mounted to a proximal end of the outer sheath for extracorporeal control of the delivery assembly.

8. The fastener applicator according to claim 7, wherein the fastener assembly includes:
a body portion;
a central shaft rotatably disposed within the body portion, the central shaft defining an elongate slot extending along the length thereof for receiving a tang of a helical fastener therein, the central shaft defining a recess formed in a proximal end thereof for selectively receiving a distal end of a drive shaft therein; and
a coil disposed between the body portion and the central shaft configured to operatively engage a coil of a helical fastener.

9. The fastener applicator according to claim 8, wherein the expandable portion includes:
a proximal hub and a distal hub; and
a plurality of support members extending between and inter-connecting the proximal and distal hubs,
the expandable portion having a un-expanded condition wherein the proximal and distal hubs are in relative spaced relation to one another and the support members in relative close proximity to one another, and an expanded condition wherein the proximal and distal hubs are in relative close proximity to one another and the support members radially expand to define interstitial regions therebetween.

10. The fastener applicator according to claim 9, wherein the drive assembly is rotatable relative to the expandable portion, and the applicator head assembly is pivotable between an orientation axially aligned with a longitudinal axis of the yoke assembly and an orientation perpendicular to the longitudinal axis of the yoke assembly.

11. The fastener applicator according to claim 10, wherein the yoke assembly includes:
a body portion having a pair of elongate slots formed on either side thereof, the applicator head assembly being operatively disposed within the body portion of the yoke assembly;
a pivot pin extending through the body portion and the applicator head assembly, at a location adjacent to the elongate slots;
a clevis operatively disposed within the body portion of the yoke assembly, the clevis including:
a pair of spaced apart arms; and
a cam pin extending from each arm of the clevis and slidably engaging a respective elongate slot of the body portion, each cam pin slidably engaging a cam slot formed in the applicator head assembly;
a driver operatively connected to the clevis, the drive including:
a coupling member configured to selectively engage a shaped end of the drive screw and a shaped end of a second bevel gear; and
an elongate body portion extending from the coupling member and extending through an aperture formed in a base portion of the clevis; and
a pusher operatively engaged with the base portion of the clevis and including a body portion defining a lumen configured to receive the elongate body portion of the driver.

12. The fastener applicator according to claim 11, wherein the applicator head assembly includes:
a housing, the housing defining a cam slot formed on either side thereof, each cam slot being configured to receive a respective can pin of the yoke assembly, each cam slot including:
a first portion substantially aligned with a longitudinal axis of the housing; and
a second portion substantially perpendicular to the longitudinal axis of the housing; and
a drive train operatively disposed in the housing.

13. The fastener applicator according to claim 12, wherein the drive train of the applicator head assembly includes:
a drive shaft having a distal end configured to selectively receive a tang of a helical fastener, and a non-circular transverse cross-sectional profile; and
a drive screw slidably disposed on a proximal end portion of the drive shaft, the drive screw includes a helical thread configured to engage threads formed in the housing, and a shaped proximal end configured to selectively engage the coupling member of the driver of the yoke assembly when the applicator head assembly is in the loading position.

14. The fastener applicator according to claim 13, wherein the drive train further includes a beveled gear system, the beveled gear system includes:
a first beveled gear co-axially aligned with and slidably supported on the drive shaft of the drive train, the first beveled gear includes a shaped central lumen configured to complement the cross-sectional profile of the drive shaft of the drive train;
a gear support box slidably and rotatably supported on the drive shaft, the gear support box including a stub extending therefrom; and
a second beveled gear rotatably supported on the stub of the gear support box and operatively engaged with the first beveled gear, the second beveled gear includes a shaped end configured to selectively engage the coupling member of the driver of the yoke assembly when the applicator head assembly in is the firing position.

15. The fastener applicator according to claim 14, wherein the applicator head assembly includes:
an ejection head operatively positioned on a distal end portion of the drive shaft, the ejection head includes a central lumen defining a helical thread formed at a distal end thereof and a recess formed in a proximal edge thereof; and
an ejection head stop operatively positioned within the ejection head and supported on the distal end portion of the drive shaft, the ejection head stop includes a key extending therefrom and configured for engagement in the recess formed in the proximal edge of the ejection head and a slot formed in the housing of the applicator assembly.

16. The fastener applicator according to claim 15, wherein the ejection head includes a saw-toothed distal end face.

17. The fastener applicator according to claim 16, wherein the applicator head assembly includes a coil spring supported on the drive shaft and positioned between the drive screw and the gear support box, wherein the coil spring biases the drive shaft to a proximal-most position.

18. The fastener applicator according to claim 17, wherein the pivot pin fixes the axial location of the gear support box relative to the housing.

19. The fastener applicator according to claim 18, wherein the control assembly includes:
a housing for supporting an expandable portion controller and a drive assembly controller;
an expandable portion controller configured to manipulate the expandable portion between the un-expanded condition and the expanded condition; and
a drive assembly controller configured to manipulate the applicator head assembly between loading position and the firing position, and to rotate the applicator head assembly about a longitudinal axis of the yoke assembly.

20. The fastener applicator according to claim 19, wherein the expandable portion controller includes:
a knob rotatably supported in the housing thereof;
a spool in threaded engagement with the knob;
an outer sheath having a distal end fixedly secured to the proximal hub of the expandable portion, and a proximal end fixedly secured to the spool;
a flange fixedly supported in the housing of the control assembly; and
a first inner tube extending through the spool and through the outer sheath, the first inner tube having a proximal end fixedly secured to the flange, and a distal end secured to the body portion of the yoke assembly;
wherein, as the knob is rotated relative to spool, the spool axially translates through the knob causing outer sheath to displace relative to the first inner tube thereby manipulating the expandable portion between the un-expanded condition and the expanded condition.

21. The fastener applicator according to claim 20, wherein the drive assembly controller includes:
a knob rotatably supported in the housing of the control assembly; and
a second inner tube extending through the first inner tube, the second inner tube including a proximal end fixedly secured to the knob of the drive assembly controller, and a distal end operatively connected to the body portion of the pusher of the yoke assembly, wherein rotation of the knob of the drive assembly controller manipulates the applicator head assembly between the loading position and the firing position.

22. The fastener applicator according to claim 21, wherein the drive assembly controller further includes:
a drive handle rotatably and translatably supported on the hosing of the control assembly; and
a drive shaft extending through the second inner tube and the body portion of the pusher, the drive shaft including a proximal end fixedly connected to the drive handle and a distal end fixedly connected to the body portion of the driver of the yoke assembly;
wherein as drive handle is translated relative to the housing of the control assembly the driver of concomitantly translated to selectively engage and disengage the coupling member of the driver with the shaped proximal end of the drive screw and the shaped end of the second bevel gear; and
wherein when the coupling member is engaged with the shaped proximal end of the drive screw or the shaped end of the second bevel gear, rotation of the drive handle results in rotation of the drive screw or the second bevel gear.

23. The fastener applicator according to claim 22, wherein rotation of the drive screw or the second bevel gear results in rotation of the drive shaft.

24. The fastener applicator according to claim 23, further comprising a cap operatively connectable to a distal end of the fastener cartridge assembly, the cap including a lumen having a distal end extending through a distal end of the cap and a proximal end extending through a side of the cap, wherein the lumen is configured and dimensioned to slidably receive a guide wire.

25. A method of endoluminally fastening a prosthetic graft to a vessel comprising the steps of:
providing an endovascular fastener applicator for endoluminally fastening the prosthetic graft to a vessel at the aneurysm site, the endovascular fastener applicator including:
a delivery assembly configured for positioning within a vessel, the delivery assembly including:
an expandable portion disposed adjacent a distal end of an outer sheath and being expandable to support the prosthetic graft in contact with an inner surface of the vessel;
a yoke assembly disposed within the expandable portion;
an applicator head assembly pivotably mounted to the yoke assembly and movable between a loading position longitudinally aligned with the yoke assembly, and a firing position oriented substantially perpendicular to the yoke assembly; and
a fastener assembly positioned at a distal end of the expandable portion, the fastener assembly retaining at least one fastener therein for loading onto the applicator head assembly when the applicator head assembly is in the longitudinally aligned position; and
a control assembly mounted to a proximal end of the outer sheath for extracorporeal control of the delivery assembly;
positioning a prosthetic graft at an aneurysm site;
positioning the delivery assembly at the aneurysm site;
radially expanding the expandable portion against an inner surface of the prosthetic graft to thereby maintain the prosthetic graft in position at the aneurysm site;
connecting the applicator head assembly to the fastener assembly;
transferring a helical fastener from the fastener assembly to the applicator head assembly;
disconnecting the applicator head assembly from the fastener assembly;
pivoting the applicator head assembly from the loading position to the firing position; and
firing the applicator head assembly and driving the helical fastener loaded thereon into the prosthetic graft.
